(19) United States
(12) Patent Application Publication
(10) Pub. No.: US 2005/0251347 A1
(43) Pub. Date: Nov. 10, 2005

(54) Automatic visual recognition of biological particles

(76) Inventors: Pietro Perona, Altadena, CA (US); Marc'aurelio Ranzato, New York, NY (US); Richard C. Flagan, Pasadena, CA (US)

Correspondence Address: GATES & COOPER LLP;HOWARD HUGHES CENTER, 6701 CENTER DRIVE WEST, SUITE 1050, LOS ANGELES, CA, 90045, US

(21) Appl. No.: 11/122,575

(22) Filed: May 5, 2005

Related U.S. Application Data
(60) Provisional application No. 60/568,575, filed on May 5, 2004.

(51) Int. Cl. G06F 19/00; G01N 33/48; G01N 33/50

(52) U.S. Cl. 702/19; 382/128

(57) ABSTRACT

A method and system provide the ability to automatically recognize biological particles. An image of biological particles (e.g., airborne pollen or urine) is obtained. One or more parts of the image are detected as containing one or more particles of interest. Feature vector(s) are extracted from each detected part of the image. Non-linearities are applied to each feature vector. Each part of the image is then classified into a category of biological particle based on the one or more feature vectors for each part of the image.

### CROSS-REFERENCE TO RELATED APPLICATIONS

This application claims the benefit under 35 U.S.C. Section 119(e) of the following co-pending and commonly-assigned U.S. provisional patent application(s), which is/are incorporated by reference herein:

Provisional Application Ser. No. 60/568,575, filed on May 5, 2004, by Pietro Perona, entitled “AUTOMATIC VISUAL RECOGNITION OF BIOLOGICAL PARTICLES,” attorneys' docket number 176.26-US-P1 /CIT-4097-P.

### STATEMENT REGARDING FEDERALLY SPONSORED RESEARCH AND DEVELOPMENT

The invention was made with Government support under Grant No. ERC: EEC-9402726 awarded by the National Science Foundation. The Government has certain rights in this invention.

### BACKGROUND OF THE INVENTION

(Note: This application references a number of different publications as indicated throughout the specification by one or more reference numbers within brackets, e.g., [x]. A list of these different publications ordered according to these reference numbers can be found below in the section entitled “References.” Each of these publications is incorporated by reference herein.)

1. Field of the Invention

The present invention relates generally to an automatic visual recognition system for biological particles. In particular, the invention provides for the recognition of particles that are found in microscopic urinalysis and airborne pollen grains. However, the invention's approach is general, segmentation free, and able to achieve a very good performance on many different categories of particles. For these reasons, the system is suitable to be used for recognition of other kinds of biological particles.

2. Description of the Related Art

Microscopic Analysis of Biological Particles

Microscopic analysis is used in many fields of technology and physics, for example in aerobiology, geology, biomedical analysis, industrial product inspection. Basically, the input image has a resolution of about 1024×1024 pixels while objects of interest have resolution of about 50×50 pixels. In all these applications, detection and classification are difficult, because of the poor resolution and maybe strong variability of objects of interest, and because the background can also be very noisy and highly variable. The present invention focuses on recognition of biological particles and especially on recognition of airborne pollen and on recognition of particles that can be found in urine. Since the invention's approach is general and performance successful on many categories of different kinds of corpuscles, the invention and its results may be naturally applicable to other many kinds of biological particles found in microscopic analysis. The invention provides a recognition system that is not a specific case study, but is segmentation free and can be easily updated to deal with new classes. To better understand the invention, a description of airborne pollen recognition and urinalysis, why and how such analyses are performed, what the problems of manual analysis are, and the need to automate this process are useful.

Measuring Airborne Pollen Level in Air

Estimates from a skin test survey suggest that allergies affect as many as 40 to 50 million people in the United States of America. Allergic diseases affect more than 20% of the U.S. population and are the sixth leading cause of chronic disease. In the United States, the estimated overall costs for allergic rhinitis in 1996 totaled 6 billion dollars and two years later, the increased absenteeism and reduced productivity due to allergies cost to companies more than 250 million dollars [1]. Moreover, from 1982 to 1996, the prevalence of asthma increased to 97% among women and 22% among men. These statistics are an example that perfectly corresponds to the situation and trend in all other countries in the world. An allergy is an abnormal reaction to an ordinarily harmless substance called allergen, [1] [27]. When an allergen is absorbed into the body of an allergic person, the person's immune system views the allergen as an invader and a chain of abnormal reactions begins. The effects of this response are runny nose, watery eyes, itching and sneezing. People with these symptoms are unable to work and even to sleep. The most common allergens are: pollen particles, molds, dust mites, animal dander, foods, medications, and insect stings. Pollen grains are clinically the most important outdoor allergens and the most allergenic species are: American elm, paper birch, red alder, white oak, white ash, olive, mulberry, pecan, black walnut, sycamore, grass, chenopod, rumex, and plantago. Note that asthma and allergies can affect anyone, regardless of age, gender, and race.

In order to make forecasts and to aid in the diagnosis, treatment and management of allergic diseases, many stations with air sampling equipment are spread in the territory to collect airborne pollen particles. The most popular device is the volumetric spore trap, see FIG. 1, which draws air into the sampler at a given rate through a little opening. FIG. 1 illustrates a Burkard volumetric spore trap that may be used to collect airborne pollen grains and fungi. The particles in the air land on an adhesive coated microscope slide attached to a slowly rotating wheel. After a period of sampling, for example one day but most usually one week, the pollen grains caught on the sticky slides are counted under the microscope by simply cutting the long slide into several pieces. The analysis at the microscope is performed by trained observers (aerobiologists) who count and classify pollen grains. This method is slow, expensive, and inaccurate. First of all: the response time is inadequate for many applications. In a typical installation, pollen particles are collected during the week on sticky tape. The tape is analyzed only once a week. The results of such analysis are therefore sometimes available one week after the fact, rendering them useless for preparations of medical response in hospitals. Second, the analysis of one weekly tape takes up to 8 hours of work by a skilled professional, thus, the yearly cost of measuring pollen contents in the air at one location could approach $30,000, too expensive for many institutions and too expensive to allow fine spatial sampling of air pollen contents. For example, the National Allergy Bureau, section of the American Academy of Allergy, Asthma and Immunology's Aeroallergen Network, is the institution responsible for reporting current pollen count to the media and it has only 75 counting stations throughout the US and its members are all volunteers; if you check the website of AAAAI [1], the pollen counts are never updated to the last week!

The most important problem with the use of the above-described prior art is that the reliance on humans produces inaccurate measurements. Such inaccuracies result from two primary reasons: first, the process is tedious and it is well documented that the attention of a human operator tends to flag after 30 minutes on a demanding repetitive job; second, in order to accomplish the task at all, human operators sample coarsely the collected tapes. Measurements are thus accurate for high pollen counts, but inaccurate for low pollen counts, and even more inaccurate when estimating the concentration of pollen grains over time. In this regard, it is entirely possible to miss the presence of a given pollen in the air if the critical time when it was released is not sampled by the operator. Moreover, it is difficult to provide accurate pollen levels for areas not near to a counting station and so the actual counts are useless for most of the physicians.

Thus, to summarize, there are many reasons to justify the recent strong interest and the need to automate the count and identification of airborne pollen particles. The manual collection and analysis is not adequate because it is too slow, too expensive, not precise and not able to cover all of the territory.

Urinalysis

Urinalysis can reveal diseases that have gone unnoticed because they do not produce striking signs or symptoms. Examples include diabetes, mellitus, various forms of glomerulonephritis, and chronic urinary tract infections [2]. There are three kinds of urinalysis: macroscopic, chemical, and microscopic. Macroscopic urinalysis is a direct visual observation to assess the color and cloudiness. Chemical urinalysis uses a dipstick to determine pH, specific gravity, content of proteins, glucose, etc. and is based on the color change of the strip and on a comparison of the strip color to a color chart. The microscopic urinalysis requires a light microscope and is the most complex. A sample of well-mixed urine is centrifugated in a test tube, and then, a drop of sediment is poured onto a glass slide for the examination. Using low magnification, a well-trained expert identifies crystals, casts, squamous cells and other “large” objects. After another adjustment of the microscope to get a higher magnification, the expert can count the number of smaller objects like bacteria and red blood cells.

Particles in urine can be classified into the following 12 categories: red blood cells, white blood cells, bacteria, hyaline casts, pathological casts, crystals, squamous epithelial cells, non squamous epithelial cells, yeasts, white blood cell clumps, sperm and mucus. The microscopic analysis is very useful and generally required because it is non-invasive and provides several indications about disease progress and therapeutic efficacy, [5]. However, microscopic analysis, if manually performed, is intrinsically not precise, time consuming and expensive. Further, there is no standardization in the process of taking a volume of fluid, there is no reliability of the result because the experts may have a different training and experience, and the work may be annoying because it is repetitive and difficult. Such difficulty results from the strong similarity among some categories of particles and in the variability existing among corpuscles belonging to the same family. Moreover, this process is slow and expensive for hospitals.

For the above reasons, an automatic recognition system is required in several situations (especially when many specimens have to be analyzed). Some prior art systems for automatic recognition of particles in urinalysis are currently sold. For example, an interesting machine using a computer vision approach is made by IRIS™, a company in the field of biomedical analysis. A urine database used and described may be provided by IRIS™.

In view of the above, the manual analysis of urine is not efficient in terms of precision, cost and time, and because the automatic recognition can be improved. Below, a description of the IRIS™ system (including some of its flaws) is provided. Other systems using different techniques, such as analysis of particle refraction when these particles are hit by a laser beam, have also drawbacks because of their suboptimal performance and the difficulty to verify analysis outcomes.

Both aerobiology and urinalysis need automation. First, manual analysis is slow. For instance, the pollen level in the air is usually given in the following week and so, physicians have available this information when by this time it is too late. Second, manual analysis requires very skilled experts with a high cost for the community and the institutions. Third, because this kind of work is repetitive and difficult, results may be not accurate. Experts' results depend on their experience, their subjective judgment, their way to set up the system. There is a need for standardization in the measurements that can be achieved only with an automatic system. On the other hand, today there is no completely automatic system to detect and identify pollen particles and the machines for urinalysis are deficient in many respects. Also, visual recognition of particles in microscopic images and more generally, recognition of small object categories in images with poor resolution and contrast is a field of research that lacks significant resources and dedication.

### SUMMARY OF THE INVENTION

The invention provides an automatic visual recognition system for biological particles. In particular, the invention recognizes particles that are found in microscopic urinalysis and airborne pollen grains. The approach is general, segmentation free, and suitable to use for recognition of other kinds of biological particles.

Images of biological particles are input into a automated system of the invention. The analysis proceeds in two parts—detection and classification. In the detection stage, the system selects those parts of the image that are likely to contain a particle of interest (e.g., a pollen). The second stage (i.e., classification) consists of taking image portions that contain a visual even associable to a particle of interest, and attributing such an event either to one out of a number of known species or, to an “unknown object” category.

The classification stage also extracts feature vectors from the detected parts of the image. These feature vectors are used in the classification process. In addition, the invention applies non-linearities to each feature vector that serves to significantly reduce the error rate during classification.

### BRIEF DESCRIPTION OF THE DRAWINGS

Referring now to the drawings in which like reference numbers represent corresponding parts throughout:

FIG. 1 illustrates a Burkard volumetric spore trap that may be used to collect airborne pollen grains and fungi;

FIG. 2 illustrates an outline of an automated analysis system in accordance with one or more embodiments of the invention;

FIG. 3 illustrates an outline of a classification system in accordance with one or more embodiments of the invention;

FIG. 4 illustrates twelve categories of samples of a database of cells/particles in urine in accordance with one or more embodiments of the invention;

FIGS. 5A and 5B illustrate pictures of pollen grains collected manually from flowers in accordance with one or more embodiments of the invention;

FIG. 6 illustrates a Matlab graphical user interface illustrated that may be used to collect a number of samples in accordance with one or more embodiments of the invention;

FIG. 7 illustrates examples of some pollen grains collected in accordance with one or more embodiments of the invention;

FIG. 8 illustrates an outline of an analyzer module in accordance with one or more embodiments of the invention;

FIG. 9 illustrates a Fourier Mellin transform of a bacterium image in accordance with one or more embodiments of the invention;

FIG. 10 illustrates a graphic representation of a confusion matrix of a classifier using features derived by Fourier Mellin Transform in accordance with one or more embodiments of the invention;

FIG. 11 illustrates a typical image of a database by Burl et al. in accordance with one or more embodiments of the invention;

FIG. 12 illustrates an outline of a filtering based approach system using a difference of Gaussians in accordance with one or more embodiments of the invention;

FIG. 13 illustrates an example of the involved computations using an image of pollen database in accordance with one or more embodiments of the invention;

FIG. 14 shows the plots of the Gaussian kernel and the difference of the Gaussian (DoG) kernel and their DFT in the 1-D case in accordance with one or more embodiments of the invention;

FIG. 15 shows a synthetic example in which two filters with DoG kernels having different σ are applied to an image in accordance with one or more embodiments of the invention;

FIG. 16 shows resulting ROC curves from experiments run on a database of pollen particles captured by an air sampler machine in accordance with one or more embodiments of the invention;

FIG. 17 illustrates the masks and morphological operations computed by a morphological detector in accordance with one or more embodiments of the invention;

FIG. 18 illustrates the original image with a mask superimposed in accordance with one or more embodiments of the invention;

FIG. 19 illustrates the original image with boxes automatically detected by a method of the invention and by an expert;

FIG. 20 shows ROC curves drawn varying one threshold each time and keeping to default values the other parameters;

FIG. 21 shows an example of images of invariants in accordance with one or more embodiments of the invention;

FIG. 22 shows all the 36 invariants for a bacterium image in accordance with one or more embodiments of the invention;

FIG. 23 shows 100 images taken from each of two pollen categories with features extracted with two components (without non-linearity application) in accordance with one or more embodiments of the invention;

FIG. 23(c) illustrates a mapping of a first non-linearity studied in accordance with one or more embodiments of the invention;

FIG. 23(d) shows mapping functions in accordance with a second non-linearity applied to invariants in accordance with one or more embodiments of the invention;

FIG. 23(e) shows mapping functions of a the third non-linearity applied to invariants before averaging in accordance with one or more embodiments of the invention;

FIG. 24 shows 36 IPCs (with the highest corresponding eigenvalues) computed from the first 500 images of each class belonging to the urine database in accordance with one or more embodiments of the invention;

FIG. 25 illustrates that the first components are the most important in a data representation in accordance with one or more embodiments of the invention;

FIG. 27 illustrates a set of principal components in accordance with one or more embodiments of the invention;

FIG. 28 shows a summary of a general algorithm in pseudo-code in accordance with one or more embodiments of the invention;

FIG. 29 shows the pseudo-code of a customized version of AdaBoost.M1 in accordance with one or more embodiments of the invention;.

FIG. 30 illustrates a simple example of binary classification using kernels for different values of parameters in accordance with one or more embodiments of the invention;

FIG. 31 illustrates error correction codes used in SVM experiments to solve the multiclass classification problem in accordance with one or more embodiments of the invention;

FIG. 32 illustrates a classifier using MoG and features based on local jets in accordance with one or more embodiments of the invention;

FIG. 33 illustrates a classifier using MoG and feature based on image and spectrum principal components in accordance with one or mote embodiments of the invention;

FIG. 34 illustrates a classifier using MoG and a combination of feature in independence hypothesis in accordance with one or more embodiments of the invention;

FIG. 35 illustrates a classifier using MoG and making a combination of experts' response, first training in accordance with one or more embodiments of the invention;

FIG. 36 illustrates a classifier using MoG and making a combination of experts' response, second training (or modeling of results in dummy test) in accordance with one or more embodiments of the invention;

FIG. 37 illustrates a classifier displayed using MoG and features based on local jets in accordance with one or more embodiments of the invention;

FIG. 38 shows a classifier using MoG and feature based on image and spectrum principal components in accordance with one or more embodiments of the invention;

FIG. 39 shows a classifier using MoG and making a combination of experts' response in accordance with one or more embodiments of the invention;

FIG. 40, the training, validation and test error rates are shown at each round in accordance with one or more embodiments of the invention;

FIG. 41 illustrates a classifier using MoG and feature based on local jets in accordance with one or more embodiments of the invention;

FIG. 42 illustrates a classifier using MoG and a feature based on image and spectrum principal components in accordance with one or more embodiments of the invention;

FIG. 43 illustrates a classifier using MoG and feature combination in indep. hyp. in accordance with one or more embodiments of the invention;

FIG. 44 illustrates a classifier using MoG and making a combination of experts' response in accordance with one or more embodiments of the invention;

FIGS. 45-47 shows the average results of 100 experiments on the full data set taking randomly 10% of images for test in accordance with one or more embodiments of the invention;

FIG. 48 illustrates test and training confusion matrices for the original classifier using MoG and local jet based features;

FIG. 49 illustrates AdaBoost.M1 in accordance with one or more embodiments of the invention;

FIG. 51 illustrates a test and training confusion matrix for the six most numerous categories of “pure” pollen in accordance with one or more embodiments of the invention;

FIG. 52 illustrates a test confusion matrix for the 13 most numerous categories of “pure” pollen in accordance with one or more embodiments of the invention;

FIG. 53 illustrates a pollen database in accordance with one or more embodiments of the invention;

FIG. 55 illustrates a pollen database graphical estimate of the error rate when the training is done using many (more than 1000) images of pollen grains captured by a volumetric spore trap in accordance with one or more embodiments of the invention;

FIG. 56 illustrates a mask computation performed by a morphological detector in accordance with one or more embodiments of the invention;

FIG. 57 illustrates errors of a morphological detector in accordance with one or more embodiments of the invention;

FIG. 58 illustrates the misclassified images with the estimated class and the assigned probability (in percentage) in accordance with one or more embodiments of the invention;

FIG. 59 shows a collage in which in the central column there are some (misclassified) images of FIG. 58 with assigned high probability in accordance with one or more embodiments of the invention;

FIG. 60 shows some patches of a database in accordance with one or more embodiments of the invention;

FIGS. 61 and 62 show the proportional error rate as a function of the number of parameters when the “unknown2” class is not modeled and when it is modeled in accordance with one or more embodiments of the invention;

FIGS. 63-64 shows the averaged values of error rates for experiments run for various choices of thresholds in accordance with one or more embodiments of the invention;

FIG. 65 shows a test and training confusion matrix, left and right respectively in accordance with one or more embodiments of the invention;

FIG. 66 shows a test and training confusion matrix, left and right respectively in accordance with one or more embodiments of the invention;

FIG. 67 is an exemplary hardware and software environment used to implement one or more embodiments of the invention; and

FIG. 68 illustrates the logical flow for implementing one or more embodiments of the invention.

### DETAILED DESCRIPTION OF THE PREFERRED EMBODIMENTS

In the following description, reference is made to the accompanying drawings which form a part hereof, and which is shown, by way of illustration, several embodiments of the present invention. It is understood that other embodiments may be utilized and structural changes may be made without departing from the scope of the present invention.

Overview

One or more embodiments of the invention provide a system for automatic recognition of particle categories. Furthermore, the system provides a general approach to enable work with several kinds of corpuscles which are found in microscopic analysis. In this way, it is easy to add new classes to the already considered set and, no new customized reprogramming is required.

Generally, input images of a system have resolution of nearly 1024×1024 pixels, while the objects of interest have square bounding boxes with sides between 30 and 200 pixels. The average side is around 60 pixels. Accordingly, the system has to handle objects with low resolution. The output is the number of detected particles in each category.

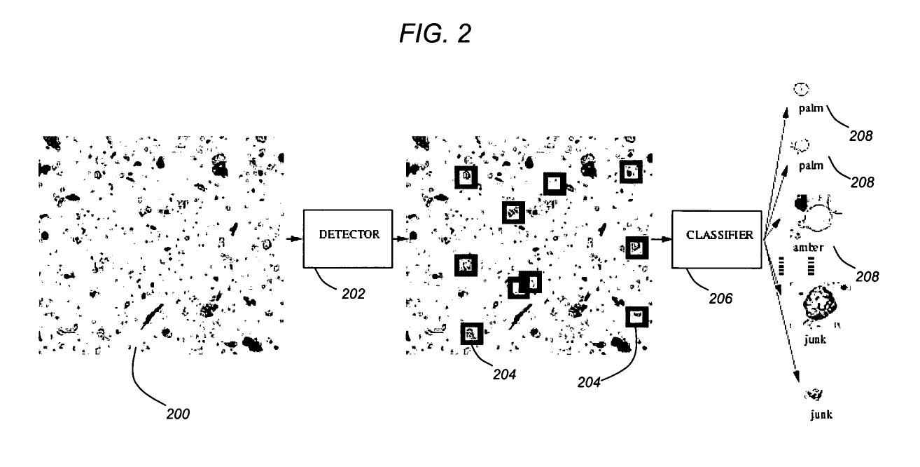

The automated analysis proceeds in two parts: (1) detection; and (2) classification as outlined in FIG. 2. In FIG. 2, detector 202 finds interesting points in the input image 200 and gives back to the classifier 206 a certain number of patches 204. For each patch 204, the classifier 206 establishes the class 208 to which the object in the foreground (of the patch 204) belongs.

In the detection stage 202, the system selects those parts of the image 200 which are likely to contain a particle of interest, for example a pollen. This process 202 is akin to visual attention in humans: most of the computation is devoted to the most promising areas of the image.

The second stage, that is classification 206, consists of taking image portions 204 which contain a visual event associable to a particle of interest, and attributing such event either to one out of a number of known species 208 or, to an “unknown object” grab-bag category.

In urinalysis, detection 202 is not a problem, because in the pictures of stream, all particles have to be analyzed (see below). Once that overlap among cells is avoided, all objects in the image will be classified. Moreover, because of the typical low concentration of particles in the fluid, particles are quite well distributed on the original image. Detection 202 for urinalysis is not a great task. For this reason, a urine database may comprise a collection of patches with particles already well centered; whereas in pollen recognition, detection 202 is still crucial, because the input images 200 of the system are pictures of sticky tape.

A given section of tape has accumulated airborne particles for many hours. Such an image 200 contains hundreds of particles like: pollen grains, dust, insect parts, etc. The detection process 202 should quickly flag the location of those particles 204 that have at least a small chance of being pollen particles. At this stage the number of false rejects, i.e. pollen particles that are not selected, must be extremely low because some species can have low counts and we have to take into account also classifier mistakes.

Classification of both urine particle patches and pollen patches has to establish if the particle is of interest, in which case the class will be also identified, otherwise, it has to be discarded. This is a visual pattern recognition problem, whose solution is conceptually simple. Given a signal s belonging to a class k, k=1, . . . K, the main stages of classification 206 are:
1. a number of features are extracted from the original signal to form the feature vector x ∈ [symbol] that is the image transformed into a pattern.
2. a decision function must be determined:

$$\begin{matrix}r \text{:} {ℝ}^{D} \to 1 , ⋯ , K \\ x ↦ r ( x )\end{matrix}$$

In many cases, a discriminant function g(x,k) is used:

$$r \text{:} x ↦ {max}_{k ∈ 1 , ⋯ , K} g ( x , k ) \vert$$

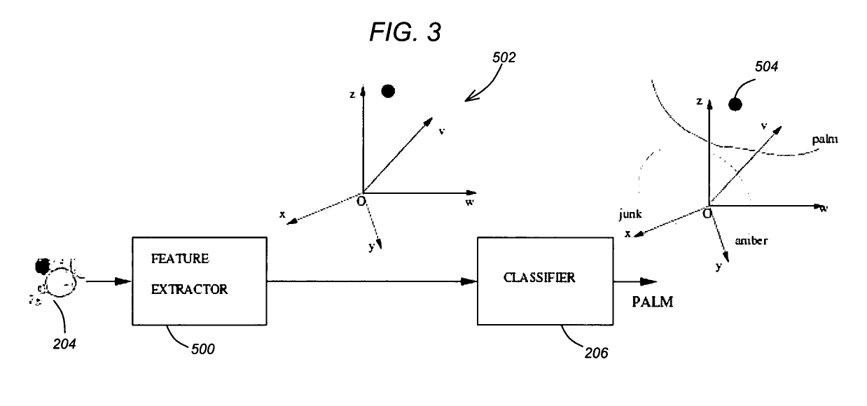

This can be regarded as a trained pattern classifier. An outline of a classification system is illustrated in FIG. 3. As illustrated, a feature extractor 500 of the system extracts a set of features 502 from an input patch 204. In the training period, the system learns how to divide the feature space. In the test phase, the system assigns a class 208 (e.g., palm) to the input image looking at the region where the test feature 504 falls.

In pattern recognition, a crucial step is designing informative features. If the features capture the peculiarities of the appearance of a set of objects, then, almost any classifier 206 will perform a competent job (Duda and Hart, [13]).

The task of classification 206, even if conceptually simple, is practically a hard challenge. The classifier 206 has to handle images with very low resolution and often poor contrast.

In addition, pollen identification is difficult because [27]:
there is a large number of look-alike categories;
each genus of plant has many species (i.e., the classification is among genera);
high winds may carry unusual kinds of pollen;
pollen shape and texture can be corrupted by previous impacts;
pollen shape depends on its orientation on slide; most frequently are seen in the equatorial position, in which they all appear as ovoid;
“apparently trees do not read literature and insist upon producing pollen grains with the same number of sides as some of other pollen kinds” [27];

Urine particle classification is difficult because:
some cells are semi-transparent, i.e. casts and mucus;
almost all categories have high variability in shape, texture and size; for example, there is no defined shape and size for clumps, crystals can be square, rectangular or hexagonal, white cells can have a nucleus or a uniform texture;
there is a smooth transition between particles belonging to different categories and even human experts do not agree in the classification of some casts or blood cells; for example, a “big” red blood cell is completely not distinguishable from a “small” white blood cell.

To summarize, the system for automatic visual recognition of particles is composed by a detector 202 and a classifier 206. The detector 202 is simply achieved in urinalysis, because all objects in foreground are objects of interest while in pollen recognition the detector 202 has to deal with a lot of non-pollen particles. The detector 202 must have an extremely low missed-detection rate. The classification of patches 204 passed by the detector 202, is similar in both cases. In both situations in fact, the classifier 2-6 handles images with poor resolution and contrast, and there is a strong variability between classes 208, but sometimes just a little variability within some categories.

Below, a description of a database that may be used with the invention is described. The database description if followed by various approaches developed in the field of visual recognition of particles or, more generally, visual recognition of small objects in images with low contrast and resolution are described.

The first system described is that of the IRIS™ urine analysis system Another approach is described by Dahmen et al. [11] which is compared to the present invention. In addition, the description below describes the detection problem the work of Burl et al. [8] is described relating to the identification of volcanoes in Venus' surface. The description concludes by introducing a technique for pollen recognition [24] and another technique for its identification, [14].

Database

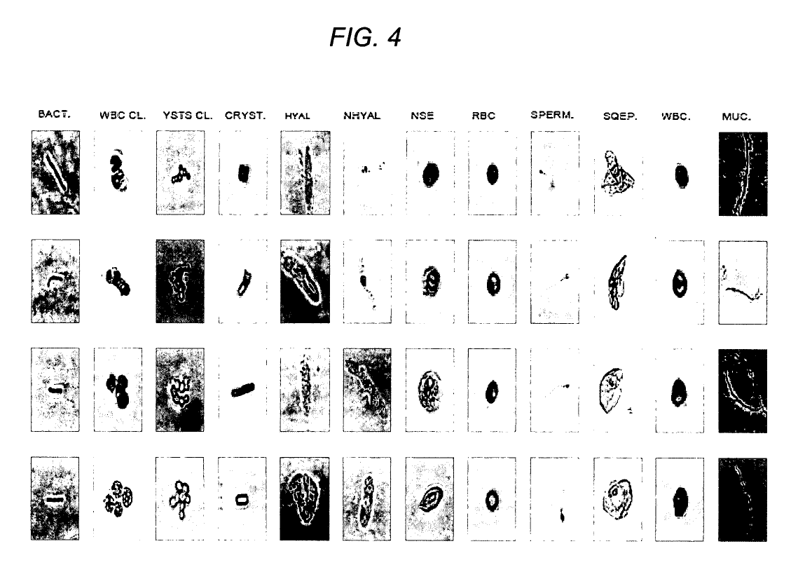

A company in biomedical analysis, IRIS™, provides a database of cells/particles in urine including: red blood cells, white blood cells, bacteria, hyaline casts, pathological casts, crystals, squamous epithelial cells, non squamous epithelial cells, yeasts, white blood cell clumps, sperm and mucus. Twelve categories of samples of this database can be found in FIG. 4. The resolution is variable and goes from 36×36 up to 250×250 pixels with 0.68 μm/pix. Table 1 shows the number of images in each class/category of the urine database.

TABLE 1

| CLASS | | NR. OF SAMPLES |
|---|---|---|
| 1. | bacteria | 1000 |
| 2. | w. blood c. | 1000 |
| | clumps | |
| 3. | yeast | 1000 |
| 4. | crystal | 1000 |
| 5. | hyal casts | 1000 |
| 6. | path. Casts | 860 |
| 7. | non squam. epith. | 999 |
| | c. | |
| 8. | red blood c. | 1000 |
| 9. | sperm | 1000 |
| 10. | squam. epith. c. | 1000 |
| 11. | white blood c. | 1000 |
| 12. | mucus | 546 |

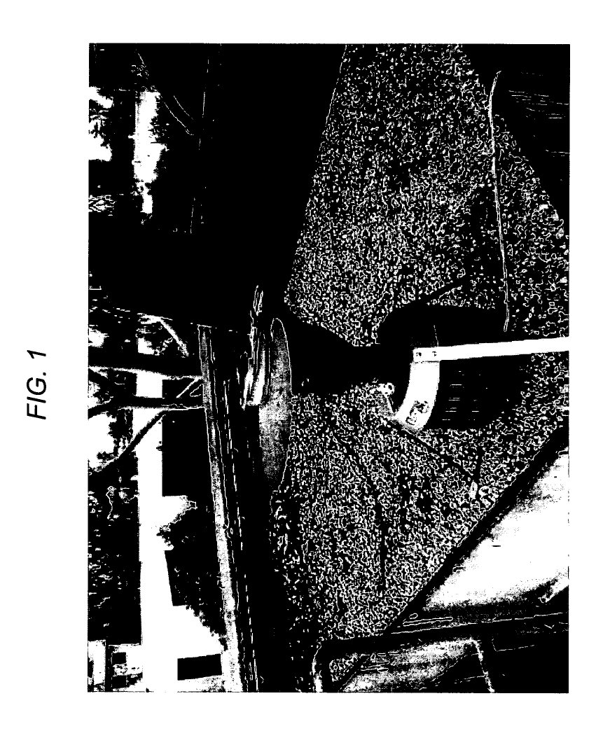

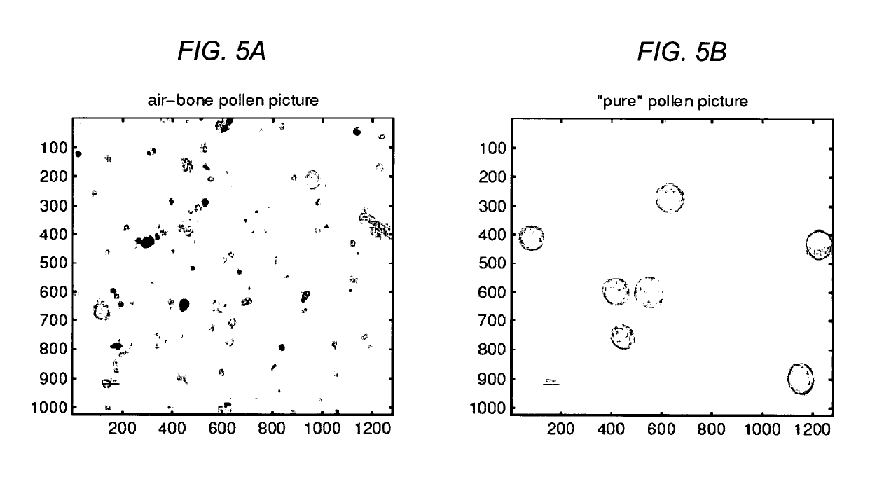

In additional database that may be used in accordance with the invention is a collection of pictures of airborne pollen particles taken with the volumetric spore trap illustrated in FIG. 1 and pictures of pollen grains collected manually from flowers, so called “pure” pollen particles. Two examples of these pictures are given in FIGS. 5A and 5B. FIG. 5A illustrates a picture of tape containing particles that are present in air (e.g., in the city of Pasadena area). FIG. 5B illustrates a picture of “pure” pollen particles. These images have a resolution of 1024×1280 pixels with 0.5 μm/pix.

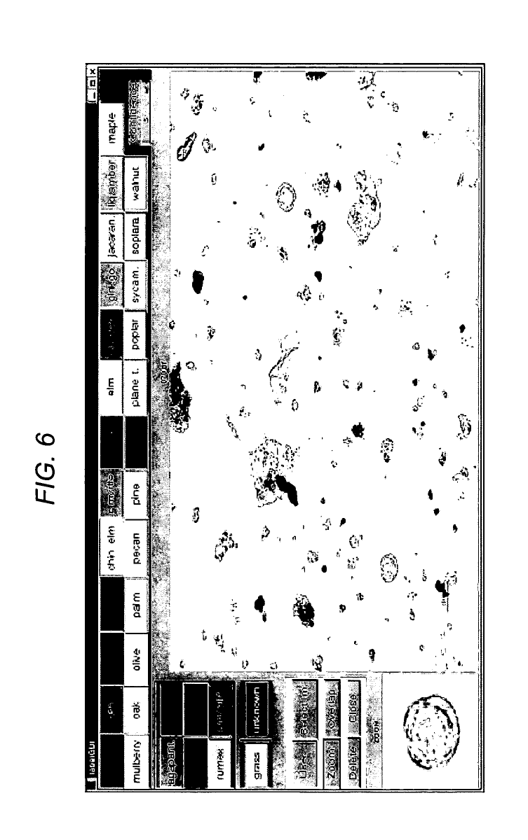

To build the database, software may enable human operators to manually collect a large training set, consisting of about 1000 samples of grains of pollen for each of 32 species which are found in a particular area (e.g., Pasadena). For example, a Matlab graphical user interface illustrated in FIG. 6 may be used to collect a number of samples summarized in Table 2.

TABLE 2

| CLASS | “PURE” | AIR SAMPLER MACHINE |
|---|---|---|
| ash | 1282 | 75 |
| chin. elm | 1916 | 124 |
| oak | 1479 | 66 |
| pecan | 1212 | 5 |
| plantain | 1549 | 0 |
| rumex | 1014 | 0 |
| birch | 342 | 0 |
| cypress | 188 | 37 |
| eucalyp. | 280 | 2 |
| grass | 285 | 2 |
| olive | 309 | 11 |
| pistac. | 314 | 0 |
| walnut | 235 | 18 |
| pine | 99 | 43 |
| liq. amber | 113 | 21 |
| alder | 78 | 13 |
| asteraceae | 0 | 6 |
| c. myrtle | 127 | 2 |
| chenopod | 0 | 20 |
| ginkgo | 0 | 3 |
| mulberry | 0 | 38 |
| palm | 0 | 18 |
| plane t. | 0 | 10 |
| poplar | 0 | 5 |
| sycamore | 0 | 16 |
| umbellif. | 0 | 4 |
| unknown | 49 | 23 |
| total | 10871 | 562 |

Using the interface of FIG. 6, the expert identifies pollen particles in the image and selects a box around each particle. The color of the box may be related to the pollen genus. The user can remove and zoom boxes, view the previous work and change the confidence of the classification. The output is a list with position and genus of each identified pollen.

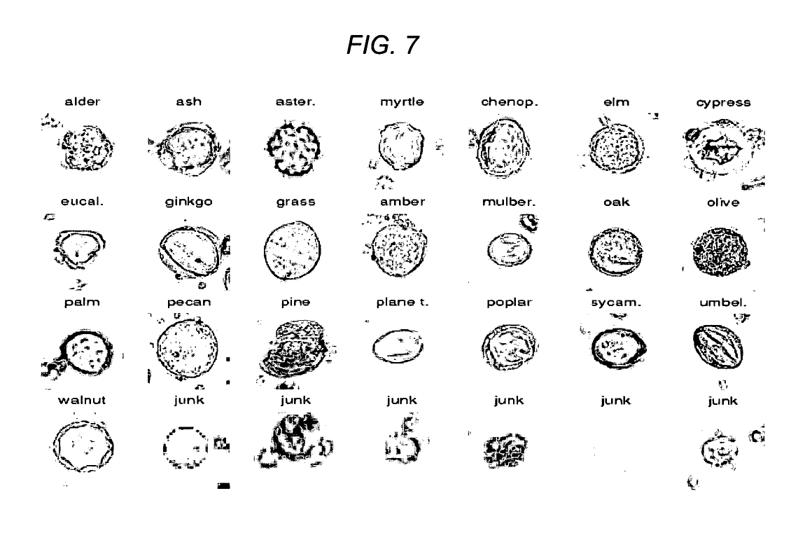

In view of the above, a reference list that stores for each image the position and the genus of each pollen identified by some expert is provided. Given this information, two databases of patches centered on pollen particles may be derived: a first database collects airborne pollen grains (see FIG. 7); the second database provides “pure” pollen grains. FIG. 7 illustrates examples of some pollen grains collected. In the last row, there are other particles that are similar to pollen grains and that are found in the acquired images. The average resolution is about 50×50 pixels. The database used in the invention has images with “pure” pollen particles because of their easier and faster labeling and because in this way it is simpler to retrieve a large number of samples which are useful to run experiments.

IRIS™ Urine Analysis System

For details of the IRIS™ system, please refer to [5] which is incorporated by reference herein. An outline of the technique offered by IRIS™ is described herein.

The iQ200 Automated Urinalysis System from IRIS™ is a bench-top analyzer able to perform chemical and microscopic analysis. The former one is focused on the determination of color, gravity and composition of urine. The latter one is used to analyze the sediment, this is useful in the diagnosis of renal and urinary tract diseases. Each kind of analysis is performed by two different but connected modules. The goal of the module for particle recognition is to overcome the limitations of manual microscopy: lack of standardization and precision, risk of biohazard, slow processing and high costs because it requires highly trained technologists. This analyzer is able to classify and quantify 12 particle categories (see the urine database described above) based on a visual recognition system.

FIG. 8 illustrates an outline of this analyzer module. The analyzer utilizes a given specimen 2 μL of centrifugated urine and produces a very thin flow through a channel. Twenty-four (24) pictures per second are taken with a CCD digital camera coupled with a microscope. In this way, the analyzer obtains around 500 images with resolution 1024×1280 and 0.68 μm/pix. Given a frame, the analyzer is able to detect patches centered on particles by morphological operations or simply by the application of a threshold. Note that the analyzer mechanically avoids the overlap among particles by regulating the thickness of the flow. Thereafter, features are extracted from each patch that take into account: the image size, the contrast of particle in foreground with respect to the background mean, the shape of the cell and its texture.

Given these features from each patch, a neural network is trained to make a decision in test. IRIS™ claims that in the every day use of their system with the data of their customers, the classifier has an error rate of about 10%. A specimen is processed in nearly 1 minute and the system allows a continuous load with a maximum of 55 samples.

IRIS claims that the hard tasks for the system are:
to handle low contrast particles because masking operations are performed to determine some features like shape and size; for this reason, the most not correctly classified classes are Mucus and Casts; and
to handle unknown particles.

In urine, there are corpuscules of unknown origin which have never been studied and which are not of interest in the actual analysis. All of these particles should be collected in an artifact class which collects also images out of focus or not well segmented. However, IRIS™ states that this class fills all the feature space and makes classification quite difficult. For this reason, IRIS™ has chosen the “abstention” rule: when the outcome of the system has a low confidence, the image is discarded and put on the artifact class. In this way, the user is shown images with high confidence and reliability for the twelve categories of interest with possibility to check mistakes (the patches are stored in the memory of two computers positioned under the bench). There is still the problem that the artifact class captures also a lot of particles belonging to other categories. In the database of the present invention, patches belonging to the artifact class may not be available.

In view of the above, a segmentation-free approach may solve the problem of misclassification among low contrast categories and boost the performance of the system. To take into account features based on shape, interest points can be extracted from a given patch. The method of the present invention is based on the first principle and achieves a very low error rate in these categories, is much simpler, is more easily updated with new classes even if it has a slightly higher global error rate.

Fourier Mellin Transform

The approach formulated by Dahmen et al. [11] is general and segmentation free. Dahmen's idea to extract features that are invariant with respect to shift, rotation and scale is powerful because it allows the same measurement to be obtained from cells that are found in different positions, orientations and scales. Dahmen aims to classify three different kinds of red blood cells (namely, stomatocyte, echinocyte, discocyte) in a database of grayscale images with resolution 128×128 pixels. Given an image, Dahmen extracts Fourier Mellin based features that are invariant with respect to 2D rotation, scale and translation. Thereafter, Dahmen models the distribution of the observed training data using Gaussian mixture densities. Using these models in a Bayesian framework a test is performed with an error rate of about 15%.

Let f[x, y] ∈ [symbol] be a 2D discrete image, its discrete Fourier Transform is defined as:

$$F ( u , v ) = \frac{1}{N} {\sum }_{j - 0}^{N - 1} {\sum }_{k - 0}^{N - 1} f [ j , k ] {ⅇ}^{- \frac{2 π ⅈ ( uj + vk )}{N}} \vert$$

With i=\sqrt{−1} and u,v=0, 1, . . . , N−1. It can be easily shown that the amplitude spectrum A of F(u,v) is invariant with respect to translation, inverse invariant with respect to scaling and variant with respect to rotation. By transforming the rectangular coordinates (u,v) of A(u,v) to polar coordinates (r,θ) and by using logarithmic scale for the radial axis, image scales and rotations become shifts in the log-polar representation Ã of A. If you compute the amplitude spectrum B of the Fourier Transform of Â, you can extract features which are invariant with respect to rotation, scale and translation. Moreover, Ã is real valued, thus B is symmetric and you can consider the values that fall in window of size for example 25×12 pixels (the origin will fall in the middle of a longer side of this window). The dimensionality of the space is reduced to d using a linear discriminant analysis (e.g., Fisher's discriminants, [6][23]).

For example, in an implementation of such a system using the above-described urine database, the images may be sampled to a common resolution of 90×90 pixels. When an image has a resolution smaller than 90×90 pixels, a frame is added with values equal to the estimated background mean. Reshaping the image in a column vector, the feature space dimension may be decreased from 8100 to 300 due to the symmetry of Fourier Mellin transform and to the windowing operation. A further reduction may be obtained by projecting the data along directions given by Fisher linear discriminants resulting in a final 11 dimensional feature space.

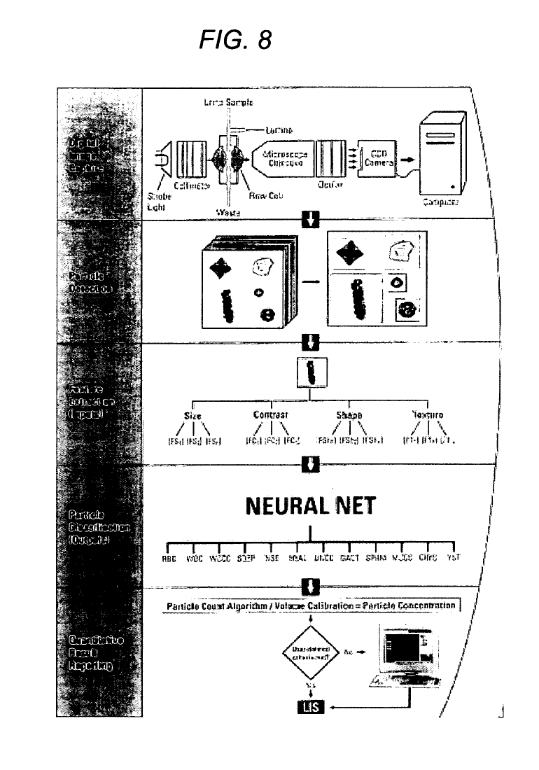

FIG. 9 illustrates a Fourier Mellin transform of a bacterium image. Note that in order to change the coordinates from rectangular to log-polar, inverse mapping must be applied and the values interpolated. If (x,y), and (r, θ) are the coordinates in the Cartesian system and in the polar one respectively, then:

$$\{ \begin{matrix}\begin{matrix}r = \sqrt{{x}^{2} + {y}^{2}} \\ θ = arctan y / x\end{matrix} & \{ \begin{matrix}x = r cos θ \\ y = r sin θ\end{matrix}\end{matrix} \vert$$

Given a grid of (r, θ) values, the correspondences in (x,y) are computed and the values in these points are taken (generally with interpolation).

With reference to FIG. 9, the plot on the upper left corner is obtained automatically by Matlab command “cart2pol” to Fourier Transform the image. The plot on the upper right corner is a mesh of the data computed by inverse mapping. The left lower corner is the magnitude of the Fourier Transform in log-polar coordinates. The symmetry of the Fourier Mellin Transform may be seen in the lower right corner wherein the superimposed superimposed shows the window of features that will be extracted.

Once a Gaussian mixture model is estimated for each class, to classify an observation x ∈ [symbol] the Bayesian decision rule is applied
xr(x)=argmaxₖp(k)p(x|k)
where p(k) is the a priori probability of class k, p(x|k) is the class conditional probability for the observation x given class k and r(x) is the classifier decision.

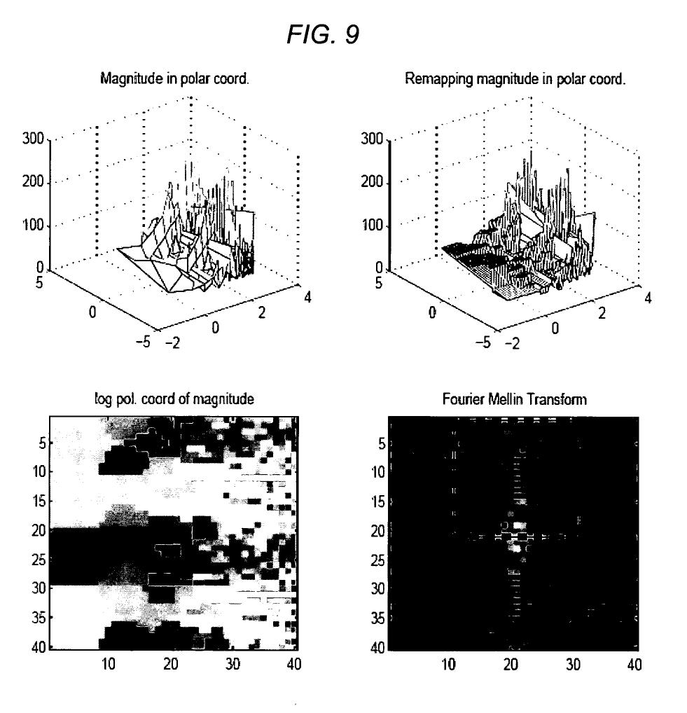

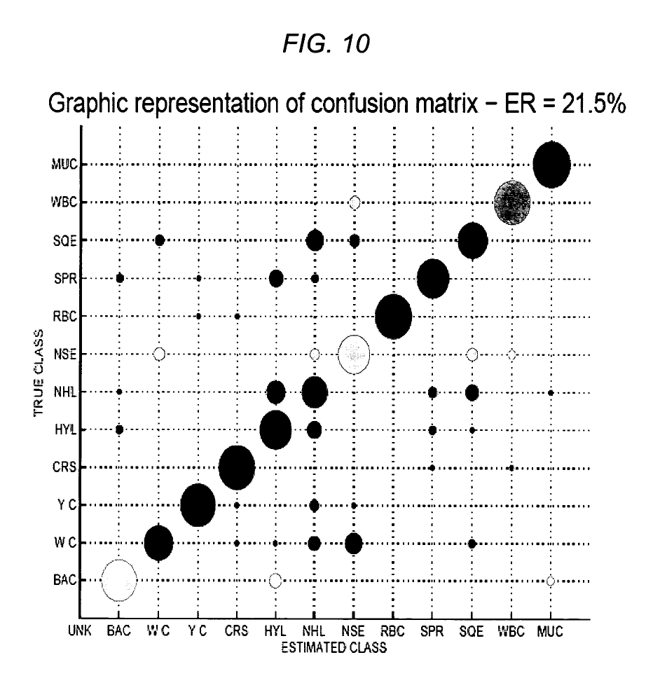

The results of this technique on the above-described database are good with an error rate of about 21% for the classification of the 12 categories of the urine database. FIG. 10 illustrates a graphic representation of the confusion matrix of the classifier using features derived by Fourier Mellin Transform. This result has been achieved based on the implementation of the method described by Dahmen et. al. [11]. Each row indicates how a given class of corpuscles was classified. The training is performed taking 500 images in each category.

The above-described performance is consistent with the one achieved by Dahmen on their database, error rate of 15% on three classes. Using the same data, the technique of the present invention is able to halve this error rate (see description below). Accordingly, Dahmen's method is particularly interesting because it is able to achieve a good performance with quite simple features and it does not require any segmentation. However, the performance is still not acceptable for any real application and not comparable with commercial systems.

Detection of Small Objects

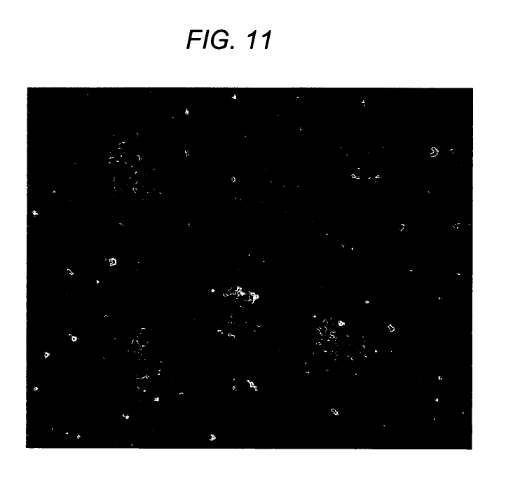

This section describes the research of Burl et al. [8] relating to the identification of volcanoes on Venus' surface. A typical image of Burl's database is shown in FIG. 11. The image shows a 30Kmx30Km region on Venus containing a number of small volcanoes. Burl's database had many thousands of 1024×1024 images derived from the successful Magellan mission to Venus of NASA-JPL (1989). The task was to identify, in this large database, the volcanoes by an automatic vision system. This specification refers to this work because the detection of these small objects in such a big image may be similar to the detection of pollen grains in a picture of sticky tape piece.

Through a graphical user interface, Burl labeled examples in a certain number of images. Applying a suitable matched filter and looking for the maximum values in the cross-correlation image interesting points are found. Given an interest point, a patch of k×k pixels centered on the interest point are considered using principal component analysis (or discrete Karhunen-Loeve transform [16]) to reduce the space dimension. Finally, the classification is achieved using a quadratic classifier, the two classes (volcano and not-volcano) are modeled with Gaussian densities and then, in test Bayes' rule is applied:

$$p ( {w}_{i} \vert y ) = \frac{p ( y \vert {w}_{i} ) p ( {w}_{i} )}{p ( y \vert {w}_{1} ) p ( {w}_{1} ) p ( y \vert {w}_{2} ) p ( {w}_{2} )} \vert$$

where y is the observed feature vector and w, is the i-th class.

The main common aspects between Burl and the present invention may include:
method to build the database (e.g., the pollen database);
the philosophy of the detector for which the present invention allows a high number of false alarms to get an high detection rate; and
the classifier philosophy because there is a modeling of training data and then a decision based on Bayes' rule.

However, the main differences include:
Burl's classifier works in a binary case (only two classes);
Burl's detector handles images with a lot of gaussian noise, compared to the (pollen database) images of the present invention that have only much more variability in the background; and
the categories of the present invention can have less variability in size but generally, have more variability in shape and texture.
   Using SEM and 3D Data for Pollen Recognition

This section describes research developed mainly by Ronneberger [24] at Freiburg University in collaboration with the weather service of Germany and Swiss. In their work, Ronneberger highlights that by using fluorescence microscopy instead of the common translucent one, it is easy to discriminate between biological and non-biological material. Moreover, Ronneberger observes that an expert has to analyze a pollen grain on different planes to be sure of his classification. For these reasons, Ronneberger utilizes the highest available quality 3D data of pollen grains by a laser scanning microscope.

Given a database in which each pollen is represented by a stack of images (one image for each scanned plane), Ronneberger computes some gray-scale invariants to avoid object-specific programming. These invariants are kernels (i.e. multiplication of two gray values belonging to pixel at a certain distance) evaluated and summed over all angles or over all the shifts. Finally, Ronneberger uses support vector machines to classify.

Ronneberger's database has a collection of 26 species of “pure” pollen particles for a total of 385 corpuscules. Performance is assessed with the technique “leave-one-out”, wherein 385 tests were performed on each pollen taking for training the rest of images in the database. An error rate of 8% was achieved. The main weak points of this work are:
it is not feasible, a laser scanning microscope is very expensive and not suitable for a real-time and low-cost instrument;
the method is cumbersome because it requires a 3D analysis;
the database is very small and therefore performance is not reliable;
“pure” pollen grains are much less variable in appearance and size than pollen particles captured by air sampler machine.
   Pollen Identification With Paradise Network

In [14], the authors describe two methods of pollen identification in images taken by optical microscope. This work is one of the first attempts to automate the process of pollen identification using optical microscope. Indeed, several researches were done using scanning electron microscope (SEM) and good results were achieved, [29] [18] [22] [19]. But, SEM is expensive, slow and absolutely not suitable for a real-time application.

Their first method is a model based approach and it assumes that pollen grains have a “double edge” in the exine. Thus, a “snake” (a proper spline) is used to detect the presence of this edge. Since not all pollen grains show the double edge property and since the achieved performance is not so good, further details are not necessary.

The second approach uses the so-called “Paradise Network”. Several small templates that are able to identify important features in the object, are generated and then linked together to form a certain number of patterns. This composition is repeated in test and the system looks for the best matching between the pollen and non-pollen patterns. The network is able to recognize nearly 80% of pollen as pollen and misclassify 4% of the debris as pollen.

This work is interesting, because images acquired with the common equipment are used, namely a volumetric spore trap and the optical microscope. On the other hand, performance is still not acceptable for any practical application and is referred to a system that receives as input already segmented images of pollen or junk particles. Moreover, the study is limited only to pollen particles with a double edge in the exine.

In view of the above, a system for an automatic system for particle recognition is needed as opposed to the manual analysis done by highly trained experts. In the urinalysis case, even if there is already a good system for automatic particle recognition, it can be still improved using segmentation free approach and trying to build a more flexible system able to extend the classification to new categories. Pollen recognition is still manually done. The attempts to use SEM provide good but useless results because no cheap, portable and real-time instrument can adopt this technology. On the other hand, no research was able to find good performance in detection and classification using optical microscope images of sticky tape pictures.

All of the above described considerations justify the present invention directed towards the automatic visual recognition of biological particles.

Detector

Detection 202 is the first stage of a recognition system. Given an input image 200 with a lot of particles, it aims to find key points, which have at least a little probability to be a corpuscle of interest. Once one of these points is detected, a patch 204 is centered on it and then, it is passed to the classifier 206 in the next stage. It is extremely important to detect almost all the objects of interest when they are rarely found in images and their number is low, as it typically happens in pollen detection.

In the present invention, points were detected in a pollen database. This database was labeled by human experts and a reference list has been built with information about genus and position of pollen particles sampled from air and now in images. Thus, performance may be evaluated by measuring how well the automatic detector 202 agrees with the set of reference labels. A detection occurs if the algorithm indicates the presence of an object at a location where a pollen exists according to the reference list. Similarly, a false alarm occurs if the algorithm indicates the presence of an object at a location where no pollen exists according to the reference list [8].

The overall performance can be evaluated computing:

$$\begin{matrix}perc . of detection = \frac{nr . of detected interest particles}{nr . of particles of interest} * 100 \\ \begin{matrix}perc . of \\ false alarms\end{matrix} = \frac{nr . of detected particles which are not of interest}{nr . of particles of interest} * 100 \vert\end{matrix}$$

More precisely, the present invention considers a box 204 centered on the detected interest point. There is a matching between this box 204 and the expert's one, if:

$$\sqrt{{( {x}_{c1} - {x}_{c2} )}^{2} + {( {y}_{c1} - {y}_{c2} )}^{2}} \leq α \sqrt{\frac{{A}_{1} + {A}_{2}}{2}} \vert$$

where x_{\dot{a}}, y_{\dot{a}} are the coordinates of the box centroid, Aᵢ is the area of the box and α is a parameter to be chosen. During experimentation, α is chosen equal to 1/\sqrt{2}.

This value and parameter are the same ones we used in the above described software developed to label pollen grains. In that situation,

$$\sqrt{{( {x}_{c1} - {x}_{c2} )}^{2} + {( {y}_{c1} - {y}_{c2} )}^{2}} \leq α \sqrt{\frac{{A}_{1} + {A}_{2}}{2}} \vert$$

is used to establish when two expert's boxes overlap and if this condition is satisfied then, the software removes the old box because there is too much overlap and the old box is interpreted as a mistake. Analogously for the detector 202, if the same condition holds, there is a matching between the automatically detected box and the expert's one.

Typically, a detection system has some parameter to tune. When one parameter is varied then, also the false alarm and detection percentages change. Thus, by varying at each time a threshold, a sequence of percentages may be computed. The resulting curve is known as receiver operating characteristic (ROC) curve.

The below description describes two detectors 202 and their performance using ROC curves.

Detector Based on Difference of Gaussians

This detector is based on a filtering approach [20] [21].

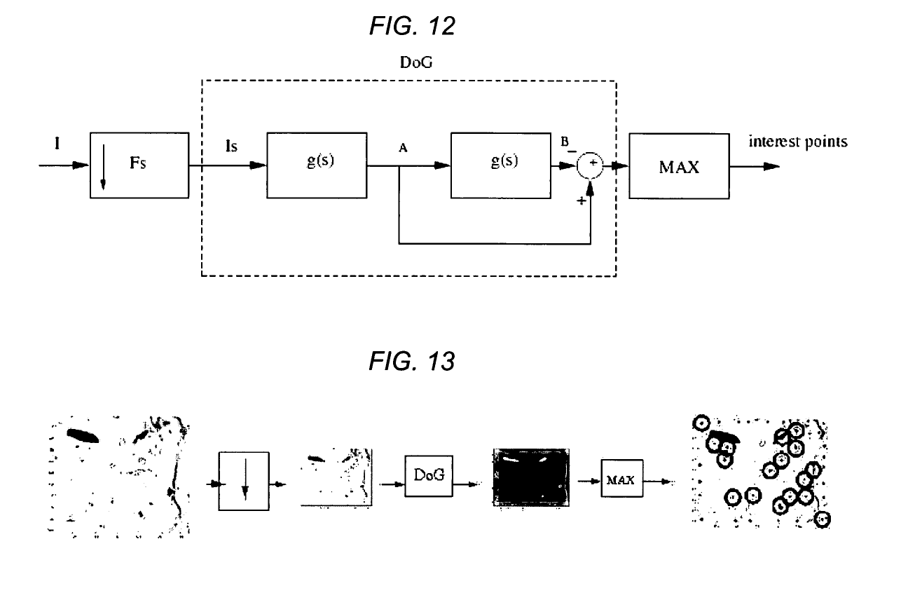

The outline of this system is shown in FIG. 12. An example of the involved computations using an image of pollen database is shown in FIG. 13.

The input image (I) is converted in gray level values and sampled with a sample frequency Fₛ. Then, it is filtered two times with a Gaussian kernel with a certain standard deviation σ to produce images A and B. Interest points are then extracted looking for the maxima and minima in the image A-B.

FIG. 14 shows the plots of the Gaussian kernel and the difference of the Gaussian (DoG) kernel and their DFT in the 1-D case. The first row illustrates basic kernels, namely the Gaussian kermel and the equivalent kernal of the whole system. The second row illustrates the DFT of previous kernels.

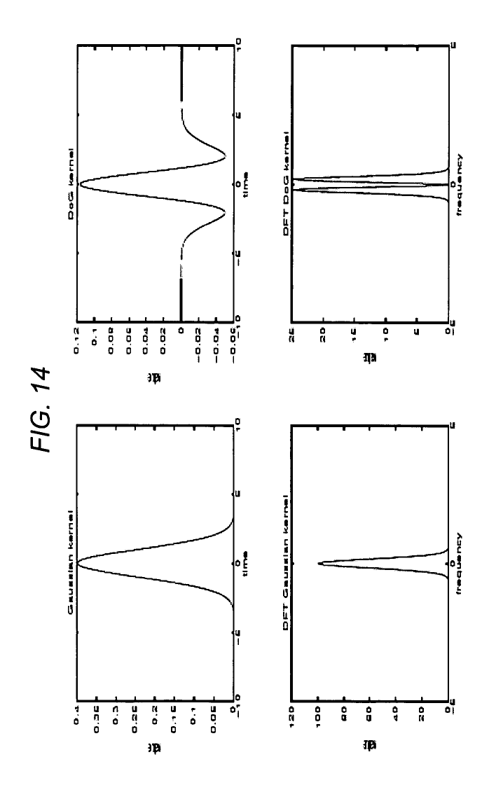

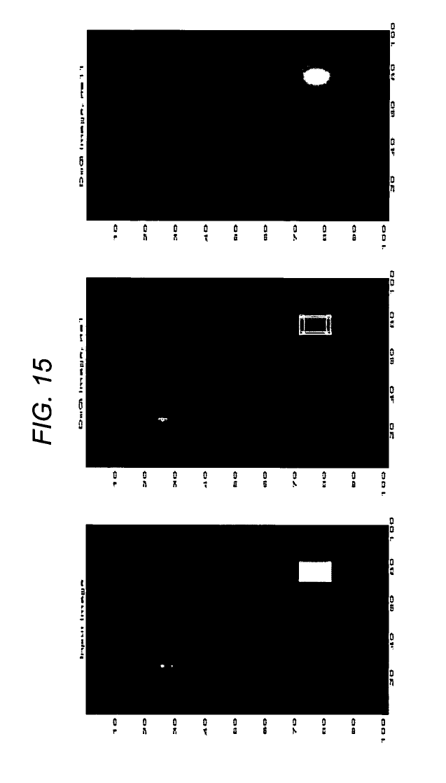

The goal of the sampling stage is to make the smallest pollen appear a little blob of few pixels. This automatically removes dust particles smaller than pollen grains and reduces the amount of computation. With a proper choice of the variance of the Gaussian kernel σ, the output image will have a peak in correspondence of round shaped objects with a certain size. FIG. 15 shows a synthetic example in which two filters with DoG kernels having different σ are applied to an image. FIG. 15 shows how the choice of scale σ is related with the dimension of objects of interest. The first image has a little blob of 1 pixel in the upper left corner and a larger one of 10 pixel diameter in the lower right corner. The second image shows a good peak in the position of the smaller blob, σ was chosen equal to 1. The third image shows a good peak in the position of the larger blob, σ was chosen equal to 8√2. Thus, when σ is low, the output image has a narrow peak in correspondence of the little blob of the input image. When σ is high, the peak is in correspondence of the largest blob. The variance of the Gaussian kernel allows the selection of the size of the interest objects.

To detect how good a point of extremum is in the output image, a paraboloid may be fit around the point. The vertex of this paraboloid gives the exact position of the interest point and its curvature is a measure of its quality. If this value is too low then, it is likely that this extremum is generated by a strip shaped object or by an object that is smaller than the one we are looking for. If this value is high then the object in that position is round and it has a size that fits the size of the target.

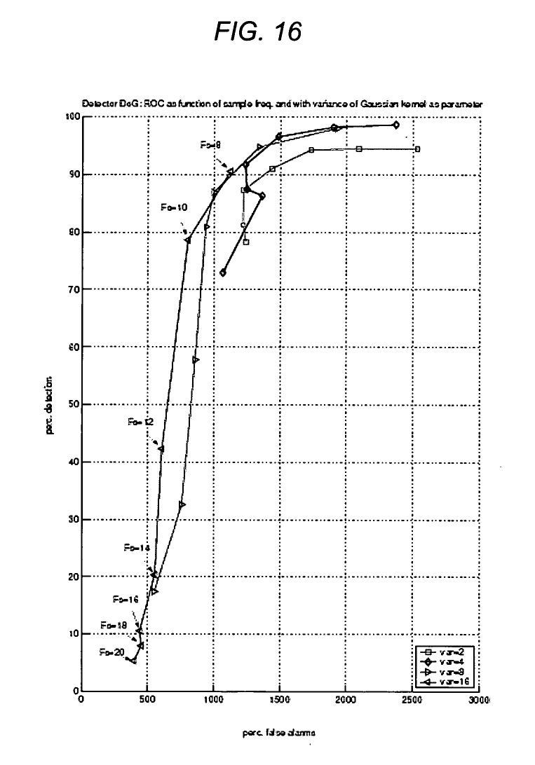

Experiments may be run on database of pollen particles captured by air sampler machine. The resulting ROC curves are shown in FIG. 16. Each ROC is drawn for many values of the sample frequency. Based on the set of values also for σ, a family of curves are produced. For example, with sample frequency equal to 12 and σ equal to 8 nearly 90% of pollen grains with 1000% of false alarms are detected.

It may be observed that the previous evaluation is optimistic because in these experiments the size of patches are kept constant. The 206 classifier needs patches 294 tightly centered on the object because around it there are other particles that can change the values of the features. Such a requirement may be confirmed by attempting to classify these detected patches and achieving poor results. Thus, additional errors for the necessary operation of box to object adaptation may be taken into account.

Moreover, in previous experiments a square box of side 200 pixels may be used because the biggest pollen, namely pine, has these dimensions. On the other hand, most pollen grains can be bounded by a box of side 50 pixels. Accordingly, it is likely to obtain some matches by chance. Because the boxes are quite big and some overlap may be admitted between them, the patches may give a match even if the interest point is not on a pollen but in some point near it.

Morphological Detector

A morphological detector is based on morphological operations on the gray level image like: edge detection, dilation, erosion, opening, closing. The need to adapt the size of the boxes around objects, encouraged attempted use of this method.

Given an image, a conversion to normalized gray level values is conducted. After the normalization, the background mean is equal to 0 and the minimum value is −

1. Thereafter:

1. the edges are computed by applying a “canny” edge detector to provide a binary image;
2. four (4) are dilated with a disk of radius 1 pixel;
3. the holes are filled;
4. the regions are labeled and a mask is computed: a region is kept if the following conditions are satisfied:
   its area is bigger than a threshold, ThresAm;
   the ratio between major and minor axis of ellipse fitting the region is less than a threshold, ThresAx;
   the mean of gray values that in the original image fall inside the region is between two thresholds, ThresMeanL and ThresMeanH;
5. two (2) are closed with a disk of radius 1;
6. one (1) is dilated with a disk of radius 1;
7. compute a new mask applying the same tests of point 4;
8. make two (2) closings and one (1) opening with the same disk of radius 1;
9. fill the holes; and
10. compute a new mask based on the previous one taking only the regions that satisfy the following tests:
   area must be between thresholds, ThresAm and ThresAM;
   the ratio between major and minor axis of ellipse fitting the region is less than a threshold, ThresAx;
   the mean of gray values that in the original image fall inside the region is between two thresholds, ThresMeanL and ThresMeanH; and
   the ratio between the area of the region and its bounding box must be grater than threshold ThresExt.

The above described series of operations may be found experimentally. However some justifications for the tests done may be used to select good regions. No pollen is too dark or too bright (see thresholds ThresMeanL and ThresMeanH), is too big or too small (see thresholds ThresAm and ThresAM), is too elongated (threshold ThresAx) or U-shaped (threshold ThresExt).

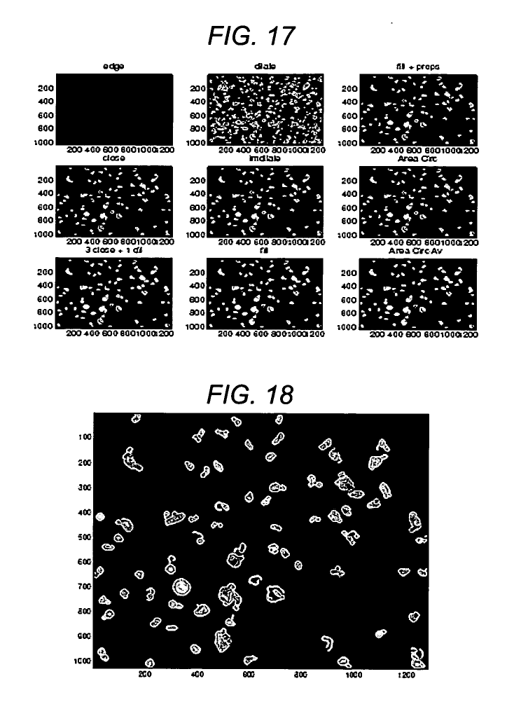

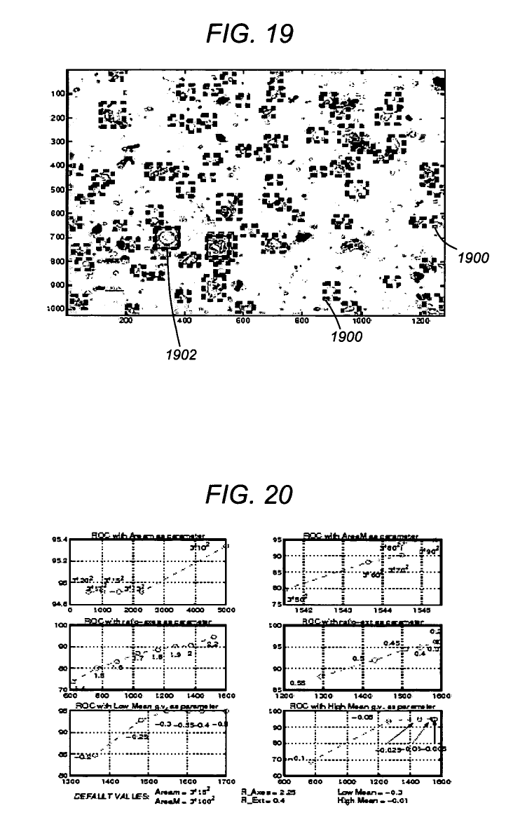

FIGS. 17, 18, and 19 illustrate the operations involved in the mask computation, the regions found and the patches that will be sent to the classifier 206. FIG. 17 illustrates the masks and morphological operations computed by a morphological detector. FIG. 18 illustrates the original image with a mask superimposed. FIG. 19 illustrates the original image with boxes automatically detected a method of the invention 1900 and by an expert 1902.

FIG. 20 shows ROC curves drawn varying one threshold each time and keeping to default values the other parameters. These curves are computed taking more than 70 images with 236 pollen grains.

A last experiment also provides a choice for these thresholds inspired by the previous ROC curves, see TABLE 3, and the result in TABLE 4 was achieved. The results illustrate a worsening when considering the full data set that may be caused by a different kind of preparation of slides (the collection of images in the experimental database took 4 months and some adjustment was done) and to the average smaller number of pollen particles per image in the other part of database.

TABLE 3
Final choice of parameters in morphological detector.

| parameter | value |
|---|---|
| ThresAream | 3 * 17² |
| ThresAreaM | 3 * 80² |
| ThresExt | 0.4 |
| ThresAx | 2.2 |
| ThresMeanL | −0.3 |
| ThresMeanH | −0.025 |

TABLE 4
Performance of morphological detector

| performance | 71 images/236 pollens | 299 images/562 pollens |
|---|---|---|
| perc. Detection | 93% | 87% |
| perc. false alarms | 956% | 1400% |

The detector 202 is able to find pollen particles with a very high probability and to do a good segmention of these particles. However, disadvantages include:
slowness because all morphological operations are made on the image with the original resolution; and
high customization, this detector is likely not suitable to detect other kinds of particles.
   Feature Extraction

In order to recognize an object in an image, it must be extracted some kind of measurements from it. These values should express the characteristics and all the information contained in the image. Moreover, we have to look for the smallest set of values with these good properties in order to speed up the recognition and to achieve more reliability. This combination of input data is called a feature and it is based on some understanding of the particular problem being tackled or by some automatic procedures, [6]. It is the heart of the classification system. If the features extracted are not representative of input images, then, no classifier will be able to do a good job.

In this section, two different kinds of features are described. The first type of feature is based on the research of Schmid et al. [9]. The second type of feature is derived from a work of Torralba et al. [28], in which they aim to discriminate between scenes containing man-made objects and scenes with natural objects. Following the descriptions of the two types of features is a description of the results of classifiers using the two sets of features.

Local Jets

In [9], the problem of matching an image in a large data set was addressed. In this method, from each interest point found in a image (using Harris' detector) a set of local gray-value invariants was computed. The features utilized in the present invention are based on these invariants, originally studied by Koenderink et al. [17].

It is desirable to extract a set of values from each image pixel, which are invariant with respect to shift and rotation. For example, it is desirable to acquire the same measurements from two images with the same rotated and shifted bacterium. Furthermore, it is desirable to get the same values when the same object is at different distance from the camera, property referred as scale invariance. Even though specific object recognition may not be of interest, the scale, shift and rotation invariance is important in the classification task. With this kind of features, it is possible to cluster together the measurements of images belonging to a certain class and acquire the same measurements, no matter the position and the orientation of the cell and these values are robust against little variations in size. Let I(x,y) be an image with spatial coordinates x and y. A local jet is defined of order N the convolution of image I with the derivatives with respect to

$$( \overbrace{x , ⋯ , y , ⋯ )}^{N times}$$

of a Gaussian kernel with standard deviation σ. The local jets are indicated with Lᵢ_{₁} _{. . . i}_{ₙ}, ∈ {x, y}| The subscripts refer to the kind of Gaussian kernel derivation. Note that the convolution of I with a derivative of Gaussian kernel is equal to the convolution of Gaussian kernel with the same derivative of 1.

In order to obtain invariance with respect to shifts and rotations, linear (or more precisely a differential) combinations of local jets are computed. The set of invariants may be limited to 9, (e.g., to a combination of third order derivatives). The first invariant is the average luminance, the second one is the square of the gradient magnitude, the fourth one is the Laplacian. These invariants are:

$$\begin{matrix}\begin{matrix}{I}_{0} = I \\ {I}_{1} = {L}_{x}^{2} + {L}_{y}^{2} \\ {I}_{2} = 2 {L}_{x} {L}_{xy} {L}_{y} + {L}_{x}^{2} {L}_{xx} + {L}_{y}^{2} {L}_{yy} \\ {I}_{3} = {L}_{xx} + {L}_{yy} \\ {I}_{4} = {L}_{xx}^{2} + {L}_{yy}^{2} + 2 {L}_{xy}^{2} \\ {I}_{5} = {L}_{xxx} {L}_{y}^{3} - {L}_{yyy} {L}_{x}^{3} + 4 {L}_{xyy} {L}_{x}^{2} {L}_{y} - 4 {L}_{xxy} {L}_{x} {L}_{y}^{2} \\ {I}_{6} = {L}_{xxy} {L}_{y}^{3} - {L}_{xyy} {L}_{x}^{3} + {L}_{xxy} {L}_{x}^{2} {L}_{y} - {L}_{xyy} {L}_{x} {L}_{y}^{2} \\ {I}_{7} = {L}_{xyy} {L}_{y}^{3} - {L}_{xxy} {L}_{x}^{3} + {L}_{xxx} {L}_{y} {L}_{x}^{2} - {L}_{yyy} {L}_{x} {L}_{y}^{2} + 2 {L}_{xxy} {L}_{x} {L}_{y}^{2} - 2 {L}_{xyy} {L}_{x}^{2} {L}_{y}\end{matrix} \\ {I}_{8} = {L}_{xxx} {L}_{x}^{3} + {L}_{yyy} {L}_{y}^{3} + 3 {L}_{xxy} {L}_{x}^{2} {L}_{y} + 3 {L}_{xyy} {L}_{x} {L}_{y}^{2}\end{matrix}$$

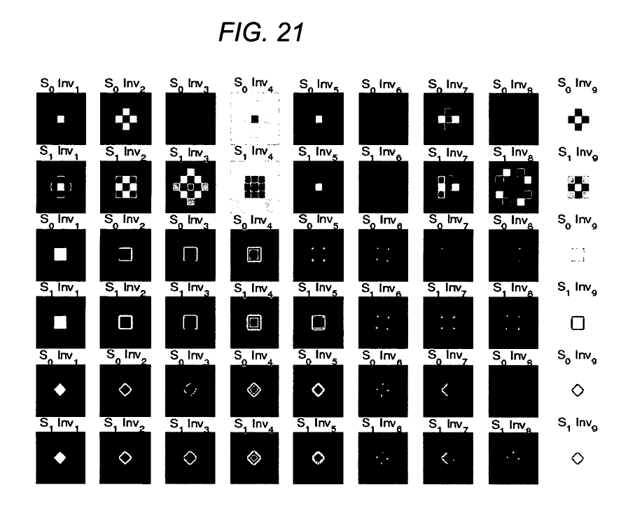

In order to deal with scale changes, these 9 invariants are computed at different scales. At each stage a blurred image (with a Gaussian kernel) is considered as starting point to derive the invariant images. In experiments, 4 scales may be considered, such that invariants are computed using the original image, its blurred version, the blurred version of this last version and so on for another stage. Totally, given an image, a stack is built with 9×4=36 images. FIG. 21 shows an example of these images of invariants at scale 0 and 1 using synthetic data: impulse (11×11 pixels) in the first and second row, square and rhombus (100×100 pixels) in the third, fourth, fifth, and sixth row respectively; the gray level values are (approximately) proportional to the invariants values. Note that the some asymmetries are due to the approximation used to represent a value in gray-level and the rotation of the square in the rhombus is not perfect due to the poor resolution used. FIG. 22 shows all the 36 invariants for a bacterium image.

Until now, an approach similar to [9 has been followed to extract from pixels feature vectors. The present invention introduces a new stage where non-linearities are applied.

Given an image of invariants, the background mean is computed from the border (a ten pixel wide frame) and the image is split into three derived images: the positive and negative part and the absolute value with respect to the background mean. Thus, each image gives 36 images of invariants that are splitted in 108 images based on this method.

Finally, an average of all the pixel values is made in each of the 108 planes and from each image, a single feature vector with 108 components is obtained.

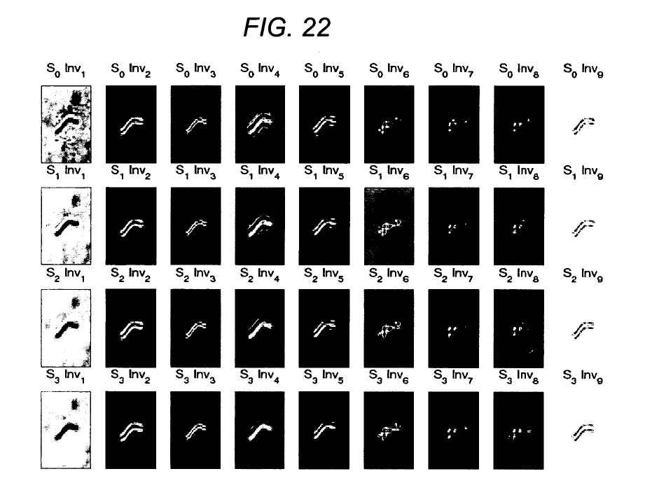

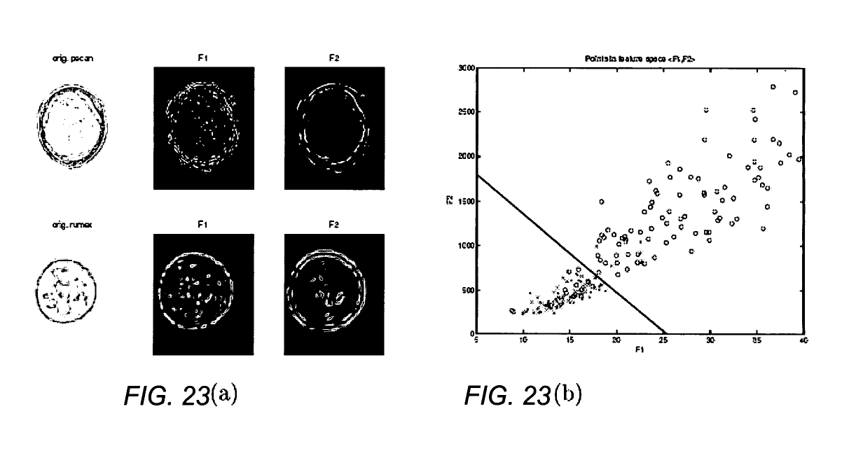

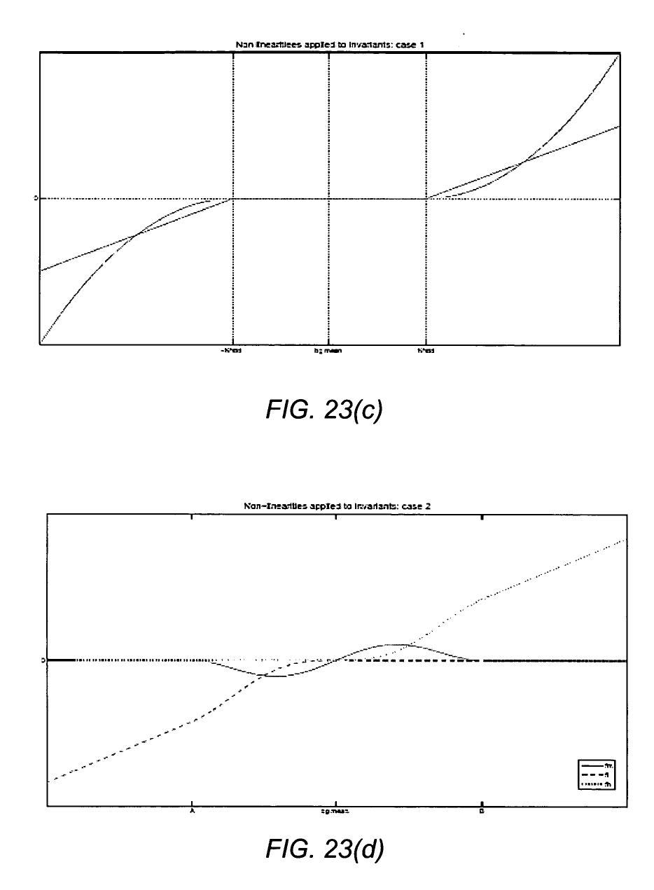

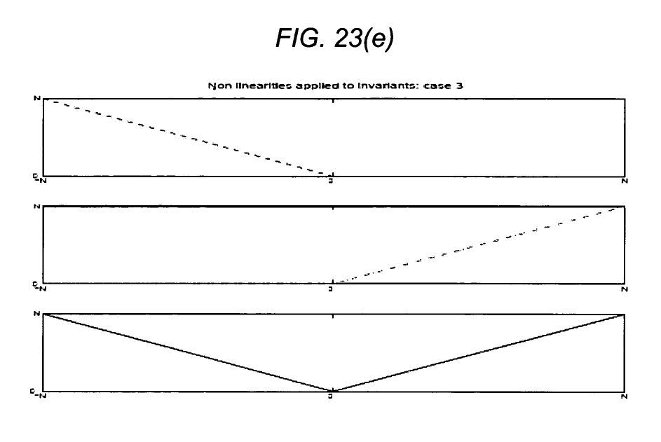

Just to have an idea of how much these features are able to separate the particles, consider FIG. 23. In FIG. 23, only 100 images are taken from each of two pollen categories and features have been extracted with only two components (without non-linearity application). However, the separation between the data is already quite good. In (a), the left column illustrates two kinds of pollen grains: pecan and rumex. The central and right columns of (a) illustrate: processing on previous images in order to get from each image a feature vector with two components. FIG. 23(b) illustrates an example of classification: points in feature space for pecan and rumex pollen, cross and circle respectively. A linear classifier is able to reach an error rate of about 15%.

It can be found experimentally that the applications of these non-linearities boosts the performance of the classifier. For example, when the system is trained on the first 500 images of each class of urine database and the nonlinearities were not applied, an error rate of 18% was achieved. However, when the nonlinearities were applied, the error rate decreased to 12%.

Average and Application of Non-Linearities

In view of the above, to get a single feature vector from an image: an average may be used taking features from each image pixel. Just to have an idea of how much these features are able to separate particles, again consider FIGS. 23(a) and (b). Again, it may be experimentally found that the performance is improved if some non-linearities are applied to the pixel features before performing the average. In this way, it may be possible to reduce the noise and to emphasize the signal. This can be interpreted as a way to do a “soft” masking avoiding any complication deriving form morphological operations. Every time a non-linearity is applied to each pixel feature the above mentioned average is computed to get the final image feature.

The first non-linearity studied is shown in FIG. 23(c). When invariants take values in a huge range then, piece-wise linear functions are applied otherwise piece-wise quadratics functions. For a given dimension, the mean and the standard deviation of the background is estimated from a 10 pixel wide frame around the border of the image. The mean is subtracted to all invariants in that dimension and then, values between the mean and a proper fraction of the standard deviation are reset to zero while the other points are transformed. More precisely, only invariants derived from the blurred image are transformed with a quadratic function. Accordingly, FIG. 23(c) illustrates a first non-linearity applied to invariants before the average: piece-wise linear and piece-wise quadratic transformation depending on the range of input invariants.

Now, a second option is described. The range of variation of the signal in each dimension is divided into three parts. Let be m the mean of the background, and σ its standard deviation. Thus, one can define:
A=m−Nσ
B=m+Nσ
where N is a parameter to be chosen. Then, each component of each pixel feature produces three values that are:

$${f}_{l} ( x ) = \{ \begin{matrix}\begin{matrix}x if x < A \\ \frac{1}{2} ( cos ( \frac{x - ( A + B ) / 2}{( B + C ) / 2 - ( A + B ) / 2} π ) + 1 ) x if A \leq x \leq m\end{matrix} \\ 0 if x > m\end{matrix} \\ {f}_{h} ( x ) = \{ \begin{matrix}\begin{matrix}x if x > B \\ \frac{1}{2} ( cos ( \frac{x - ( B + C ) / 2}{( C + D ) / 2 - ( B + C ) / 2} π ) + 1 ) x if m \leq x \leq B\end{matrix} \\ 0 if x < m\end{matrix} {f}_{m} ( x ) = x - {f}_{l} ( x ) - {f}_{h} ( x )$$

FIG. 23(d) shows the mapping functions—the second non-linearity applied to invariants before the average: ƒl is sensitive to low values, ƒₘ, to values around the background mean. ƒ_{b} is sensitive to high values.

Finally, the last transformation is described. With respect to the estimated mean value of the background, each invariant is split into three parts: positive, negative, and absolute value. In order to avoid any loss of information, the background mean is added to the last component. The mapping functions are shown in FIG. 23(e)—the third non-linearity applied to invariants before the average: each plane is split into positive, negative, and absolute value with respect to the background mean.

Note that the second and third non-linearities increase the dimension of the feature space from 36 to 108. In order to assess the performance of these features some experiments may be performed on the urine database. A classifier may be considered that models the training data with a mixture of Gaussians. Each time a different set of features is considered:
1. the ones that are computed simply averaging invariants without any non-linearity;
2. the ones that are computed applying the first non-linearity;
3. the ones that are computed applying the second non-linearity; and
4. the ones that are computed applying the third non-linearity.

For each ease, the optimal parameters have been found running experiments for many different values. The results are summarized in TABLE 4.5:

TABLE 4.5
Parameters and performance of classifiers using the
different kind of features described in this section.

| Feature | Parameters | Error rate |
|---|---|---|
| 1) averaged invariants | | |
| D | 18 | 18 |
| MoG | 4 full | |
| 2) 1^{st} non-lin. | | |
| N | 0.5 | |
| D | 8 | 16.3 |
| MoG | 4 full | |
| 3) 2^{nd} non-lin. | | |
| N | 0.5 | |
| D | 18 | 11.3 |
| MoG | 3 diag. | |
| 4) 3^{rd} non-lin. | | |
| D | 12 | 10.8 |
| MoG | 4 diag. | |

D is the number of dimension of the feature space.
MoG says how many components are in the mixture and the structure of their covariance matrix.
N is the number of standard deviation (of the background brightness) that are used to compute a certain feature (only for 1^{st} and 2^{nd} non-linearity).
Experiments may be run for D ε (8 . . . 20), N ε (0.1, 0.5. 1), covariance structure spherical, diagonal and full.
Training was performed on 480 images and tested on 20 images.
The number of components in the MoG was chosen in order to have at least three samples per parameter.

Since the third non-linearity has given the best performance, this kind of non-linearity may be referred to herein.

Decreasing the Dimensionality

In training, it is desirable to model the distribution of feature vectors belonging to all the images of a certain class with a mixture of Gaussians (MoG) or with support vector machines (SVM). However, it may not be possible to work in a 108 dimensional feature space because the number of parameters grows very quickly with the space dimensions. For example, TABLE 5 illustrates relations as a function of the choice of covariance matrix structure and as a function of the number of components in MoG.

TABLE 5
Number of parameters in MoG using N components in a D dimensional
feature space as a function of the covariance matrix structure.

| | SPHERICAL | DIAGONAL | FULL |
|---|---|---|---|
| nr. param. | N(D + 1) + N − 1 | 2ND + N − 1 | $N ( D + \frac{D ( D + 1 )}{2} ) + N - 1 \vert$ |

For instance, if D=108 and N=2, then, 220, 433, 11989 parameters are estimated in accordance to the chosen covariance matrix structure. On the other hand, a common rule of thumb says that you need at least 3 or 4 points to estimate a single parameter. Since a database may not have a huge number of images, the dimension of feature space may need to be reduced.

In order to reduce the feature space dimensionality, PCA may be applied [16] [6]. Experimentally, it may be found that linear discriminants analysis works better than PCA. Particularly, given the whole set of training feature vectors, Fisher's Linear Discriminants (FLD) may be computed [6] [23]. Suppose for simplicity to have data x ∈ [symbol] belonging only to two classes C₁ and C₂. To find the vector w such that the projected data
yₙ=w^{T}xₙ
are optimally separated. The mean vectors of the two classes are

$${m}_{1} = \frac{1}{{N}_{1}} {\sum }_{n ∈ {C}_{1}} {x}_{n} {m}_{2} = \frac{1}{{N}_{2}} {\sum }_{n ∈ {C}_{2}} {x}_{n} \vert$$

where N is the number of items in class i-th.

$${s}_{k}^{2} = \frac{1}{{N}_{2}} {{\sum }_{n ∈ {C}_{k}}}^{{m}_{k} = {w}^{T} {m}_{k}} {( {y}_{n} - {m}_{k} )}^{2} \vert$$

is then defined.

Fisher's discriminants maximize a function which is in the two-class problem,

$$\frac{{( {m}_{2} - {m}_{1} )}^{2}}{{s}_{1}^{2} + {s}_{2}^{2}}$$

that is, one attempts to maximize the distance between the means of the projected clusters and minimize the spread of the projected data. While PCA seeks the sub-space that best represents the data, FLD seeks the sub-space that maximizes the separability between the classes.

The generalization of FLD to several classes follows the same reasoning. It turns out that if one has K classes, one can have no more than K−1 directions where to project the data. For example, if it is desired to work with the 12 categories of urine database, the constraint should reduce the feature space dimension from 108 to less than 12. In order to reduce the feature space dimensions without any constraint, the data of each class may be split randomly in a proper number of sub-categories. In addition, each cluster may be divided by applying k-means algorithm [6] and estimating a MoG but without any meaningful improvement.

Image and Power Spectrum Principal Components

A feature extraction based on principal component analysis (PCA) is referred to as image and power spectrum principal components [16] [6]. Given data in a N dimensional space, the goal of PCA is to find a D dimensional subspace such that the projected data are the closest in L₂ norm (mean square error) to the original data. This means that one is looking for the subspace that provides the best representation of the original data. This subspace is spanned by the eigenvectors of the data covariance matrix having the highest corresponding eigenvalues. Often the full covariance matrix can not be reliably estimated from the number of example available, but the approximate highest eigenvalue basis vectors can be computed using singular value decomposition (SVD). Thus, if σ is the covariance matrix of the data, its SVD decomposition is

$$Σ {=}^{s υ d} {USV}^{T}$$

where if, U ∈ [symbol] V ∈ [symbol] and UU^{T}=I, VV^{T}=I S is a diagonal matrix with the elements on the diagonal (the singular values) in descending order. The first columns of U are the eigenvectors with the highest eigenvalues and can be used as basis for the original data.

In a specific case, let i(x,y) be the intensity distribution of the image along spatial variables x and y with. x, y ∈ [1, N]|es of image pixels rearranged in a column vector, then the covariance matrix is
Σ=E[(i−m)(i−m)^{T}]|
with m=E[m]. If image principal components are called IPCₙ the eigenvectors of Σ (computed by singular value decomposition) and IPCₙ(x,y) the same eigenvectors reshaped into a matrix N×N, then the following decomposition may be written:

$$i ( x , y ) = {\sum }_{n = 1}^{P} {v}_{n} {IPC}_{n} ( x , y )$$

with P≦N² and vₙ the coefficients used for describing the image i(x,y) in this new basis. Obviously, vₙ is the projection of the image along the direction given by IPCₙ.

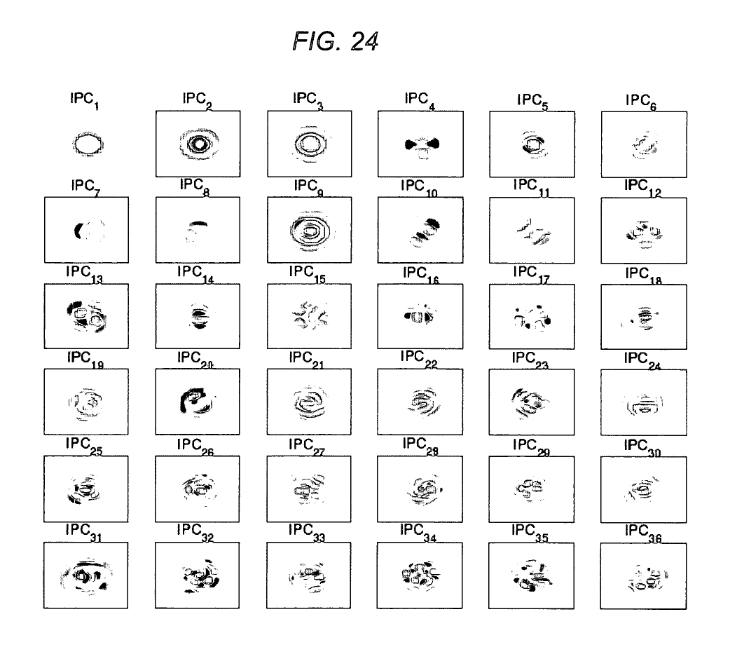

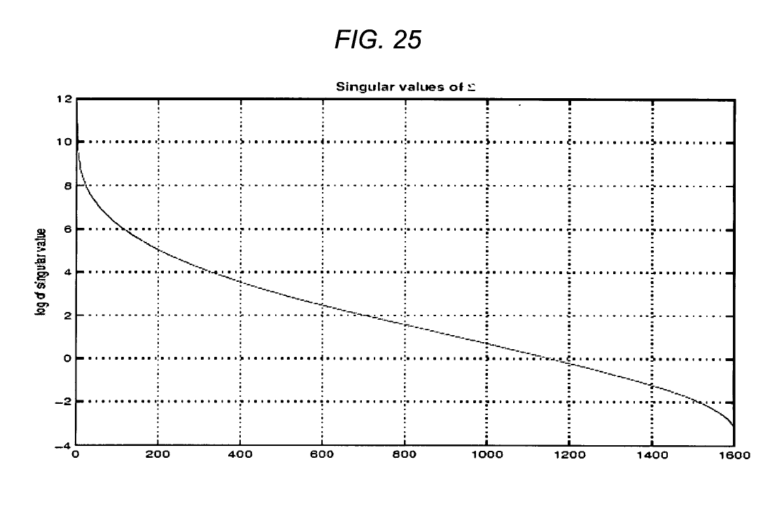

FIG. 24 shows 36 IPCs (with the highest corresponding eigenvalues) computed from the first 500 images of each class belonging to the urine database. In experiments less than 10 IPCs may be used. Each image was normalized to a common resolution of 40×40 pixels, thus the space dimension is 1600 and there are 1600 IPCs and singular values. It can be noted from FIG. 25 that the first components are the most important in the data representation. Before any computation, the background mean estimated is subtracted from the border of the image (a frame 10 pixels wide) in order to normalize the data with respect to the background brightness.

If the discrete Fourier transform (DFT) I(kₓ,k_{y}) of an image is computed, its power spectrum may be approximated with the squared magnitude of I:

$$R ( {k}_{x} , {k}_{y} ) = {\vert I ( {k}_{x} , {k}_{y} ) \vert}^{2} \vert \\ with$$

$$I ( {k}_{x} , {k}_{y} ) = \frac{1}{{N}^{2}} {\sum }_{x = 0}^{N - 1} {\sum }_{y = 0}^{N - 1} i ( x , y ) {ⅇ}^{- j \frac{2 π}{N} ( {xk}_{x} + {yk}_{y} )}$$

and fₓ=kₓ/N, f_{y}=k_{y}/N spatial frequencies. The power spectrum encodes the energy density for each spatial frequency and orientation over the whole image. PCA applied to power spectra gives the main components that take into account the structural variability between images.

First, the power spectrum is normalized with respect to its variance for each spatial frequency: R*(iₓ,k_{y})=R(kₓ,k_{y})/std[R(kₓ,k_{y})], with
{square root over (E(R(kₓ, k_{y})−E[R(kₓ, k_{y})])²]|)}

Then, PCA is applied as done before for the image, to find the spectral principal components (SPCs) and the normalized power spectrum is decomposed in

$${R}^{*} ( {k}_{x} , {k}_{y} ) = {\sum }_{n = 1}^{P} {u}_{n} {SPC}_{n} ( kx , ky ) \vert$$

FIG. 26 shows the SPCs (with the highest corresponding eigenvalues) computed from the first 500 images of each class belonging to the urine database. Each image was normalized to a common resolution of 40×40 pixels.

Until now, the basic approach has been described as presented in [28]. In one or more embodiments of the invention, a new step may be provided.

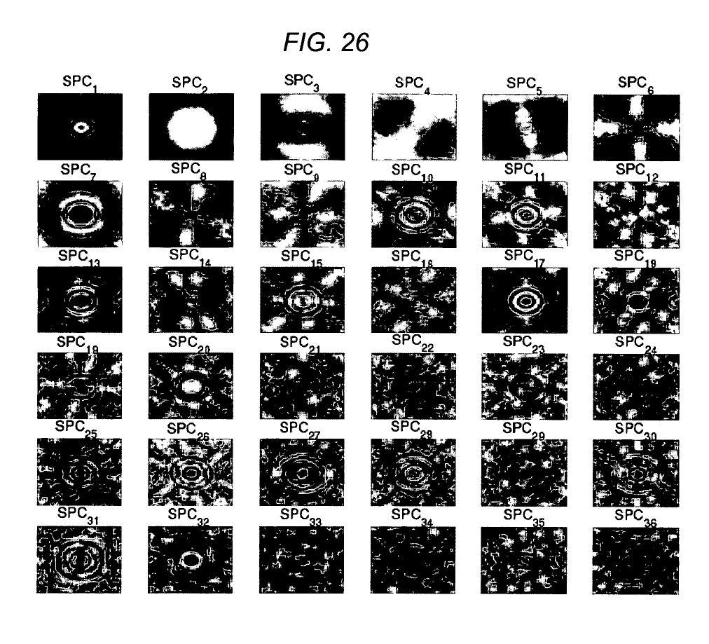

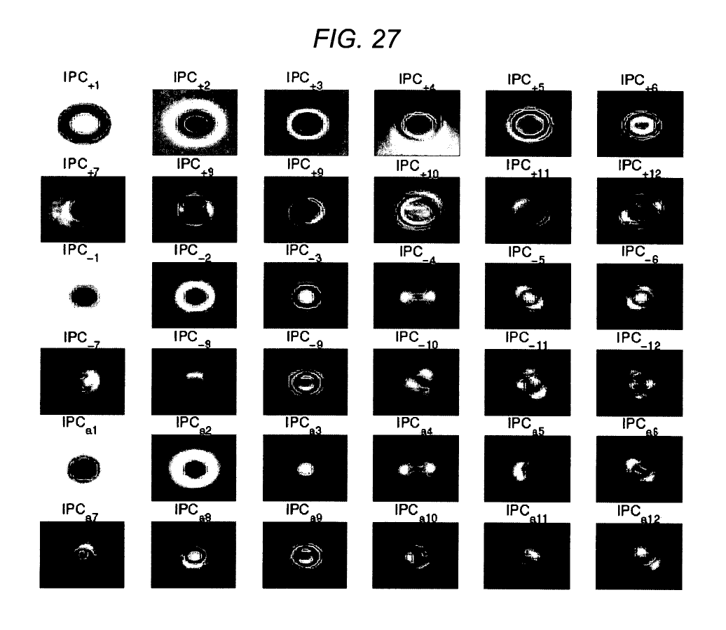

After the normalization of the image with respect to the background mean that makes this mean equal to zero, the image may be split into three derived images: the first one is its positive part I⁺, the second one its negative part I⁻ and the third one the absolute value I^{a}. This operation is equivalent to the application of three non-linear functions that are selective of high, low and mid-range responses. Then, each of these derived images is applied for the PCA to obtain IPC₊, IPC₋, IPCₐ. A set of these principal components are shown in FIG. 27 and are computed from the same data used to calculate the basis shown in FIG. 24. Thus, FIG. 27, illustrates the first 12 IPC₊, PIC₋, PICₐ derived collecting 500 images from each class in the urine database. Each image was normalized to a common resolution of 40×40 pixels.

It is experimentally proved that the classification is improved when this splitting is done; evidently, particles belonging to different categories behave differently with respect to this operation. Note that the split in positive, negative and absolute value parts can be done very efficiently. The present invention adds complexity only in the training stage because the number of PCA required is doubled (from two to four).

With this trick, the error rate is decreased from nearly 19% to 16% with reference to the classification of urine database.
N₁ⁱ,N₂ⁱ,N₃ⁱ,N^{s}

If the first coefficients computed by the projection of I⁺ along IPC₊, I⁻ along IPC₋, I^{a} along IPCₐ, and R* along SPC are considered, a feature vector from each image is obtained:

$$f = [ {υ}_{11} , ⋯ {υ}_{1 {N}_{1}^{i}} , {υ}_{21} , ⋯ {υ}_{2 {N}_{2}^{i}} , {υ}_{31} , ⋯ {υ}_{3 {N}_{3}^{i}} , {υ}_{1} , ⋯ {υ}_{{N}^{3}} ] \vert$$

where vᵢⱼ is the projection of the image Iⁱ along j-th IPCᵢ for i ∈ {+, −, a}|. How are values chosen for N₁ⁱ, N₂ⁱ, N₃ⁱ, N^{s}? In preliminary experiments, it was found that the nearly same performance can be achieved considering

$$\begin{matrix} & {N}_{1}^{i} = {N}_{2}^{i} = {N}_{3}^{i} \\ {N}^{s} = 3 {N}_{j}^{i} , & for j = 1 , 2 , 3\end{matrix} \vert$$

and applying Fisher's linear discriminants to the whole set of principal components in order to reduce the dimension of feature space to N=N₁ⁱ+N₂ⁱ+N₃ⁱ+N^{s}.

Below, the first option is considered because of its simplicity.

Interpretation

The two sets of features described above are similar, in spite of the different techniques used to compute them. Features based on PCA give the average information content of the signal at frequencies higher and higher when it is considered the projections on eigenvectors with smaller and smaller eigenvalues. Similarly, features based on invariants give the global amount of information at different bands when it is taken the average of these invariants at different scales. This is proved experimentally. The implementation of classifiers based on these features skipping the non-linearity application gave nearly the same performance, with an error rate of 20% on the twelve categories of urine database.

The application of non-linearities boosts the performance; in particular, it nearly halves the error rate of classifier using the features based on local jets.

Classification

Classification is the last stage of a recognition system. Given a patch with—hopefully—one centered object, a feature vector is extracted from the image. In training, the system learns how to distinguish among features of images belonging to different categories. In test phase, according to the test image feature, a decision is made. If the object in the image is considered a particle of interest, then its class is also identified, otherwise it is discarded because the measurement does not match any training model.

The discussion below begins with the description of training and test stage of a Bayesian classifier using Gaussian mixture models. This classifier gave the best performance with an error rate of 7.6% on urine database and 10.2% on “pure” pollen database.

However, the following descriptions present slightly different approaches. First, two ways of combining both kinds of features already described above are discussed. Second, a customized version of AdaBoost.M1, an algorithm to boost the performance of any classifier, will be defined. Finally, a classifier based on support vector machines (SVM) will be introduced.

The description below will conclude with experiments to optimize the system and some final experiments covering all the data set.

Mixture of Gaussians

This classifier is the simplest and the most powerful system developed. With the only exception of the classifier using SVM, all the other classifiers are based on it. There are two main stages of this classifier: training and test.

Training

In the training phase, one can model the distribution of feature vectors belonging to a certain class with a mixture of Gaussians (MoG) [6]. If the number of categories is K, then one has to estimate K MoGs. The estimation is done applying an expectation maximization (EM) algorithm [6].

Generally, the aim is to estimate the class-conditional distribution f(x|Cₖ), that is the probability density function (pdf) of the data, given the knowledge that they belong to a certain class Cₖ. In order to simplify the notation, the class condition may be made implicit. However, it is a straightforward generalization to consider it.

The mixture model decomposes the pdf of the data f(x) in a linear combination of M component densities f(x|j) in the form:

$$f ( x ) = {\sum }_{j = 1}^{M} f ( x \vert j ) P ( j )$$

The mixing parameters P(j), also called priors probabilities, satisfy the constraints:

$$\begin{matrix}{\sum }_{j = 1}^{M} P ( j ) = 1 \\ 0 \leq P ( j ) \leq 1\end{matrix}$$

Moreover, the conditional probability density functions verify:

$$\int f ( x \vert j ) ⅆ x = 1$$

An important property of mixture models is that they can approximate any continuous pdf to arbitrary accuracy with a proper choice of number of components and parameters of the model.

In the present invention, a mixture of Gaussians is used. Thus, the number of components and the structure of their covariance matrix must be chosen. The best choice of parameters is found experimentally. One may make use of a package for MATLAB called NETLAB in which there are functions to estimate the MoG by the application of an EM algorithm.

The goal of training is to build for each class a model of the distribution of the feature vectors belonging to the class images. This model is achieved using MoG and it describes the pdf of the class feature vectors.

Test

Given a test image, its feature vector f may be computed. Then, Bayes' rule may be applied.

The probability of the class Cₖ is given the test data P[Cₖ|x], to apply the following decision rule:
xr(x)=argmaxₖP[Cₖ|x]

The extension of Bayes' rule to continuous conditions assure that

$$P [ {C}_{k} \vert x ] = \frac{f ( x \vert {C}_{k} ) P [ {C}_{k} ]}{f ( x )} = \frac{f ( x \vert {C}_{k} ) P [ {C}_{k} ]}{{\sum }_{ = 1}^{M} f ( x \vert {C}_{j} ) P [ {C}_{j} ]} \vert$$

where f is pdf and P is probability. So, in a hypothesis of equal distribution for the class probabilities, P[Cₖ]=1/K for k=1, . . . , K, one can simplify

$$P [ {C}_{k} \vert x ] = \frac{f ( x \vert {C}_{k} )}{{\sum }_{ = 1}^{M} f ( x \vert {C}_{j} )} \vert$$

Moreover, it may be observed that P[Cₖ|x] is obtained for the same k of max f(x|Cₖ) because the factor

$${\sum }_{j = 1}^{M} f ( x \vert {C}_{j} ) \vert$$

is constant for all j. The term f(x|Cₖ) is called likelihood because it is a conditional probability density function with implicit dependence on the model parameters. Because the decision rule looks for the maximum of this term, it is referred as maximum likelihood decision rule.

A test may also be added to this framework. When training, a resolution mean and standard deviation, mᵣₖ and σₖ may be collected for each class. Then in test, the likelihood of the test data given the class Cₖ is computed only if the following condition holds:

$$r ∈ [ {m}_{{r}_{k}} - N {σ}_{k} , {m}_{{r}_{k}} + N {σ}_{k} ] \vert$$

where, r is the resolution of the test image and N is a parameter to be chosen; in experiments N=3 was chosen.

This test is mainly done to save time avoiding useless computation. If an image resolution is out of the range of every class, then it is not classified and put in an unknown class.

Combination of Features

It may be shown in the experimental part that the previous classifier using features based on principal component analysis and on local jets gives good results with an error rate below 15% on a urine database. In this section, two methods to combine these features ate presented.

If one looks at the confusion matrices of the classifier working with these features (see description and figures below), it may be noticed that a certain correlation among misclassifications suggests dependence between the two kinds of features.

However, the problem of feature combination may be simplified assuming independence between the two sets. If a hypothesis is not made between these features, the more general problem of combination of experts' response must be solved.

Independence Hypothesis

Given an image, the two techniques described above are applied to extract features based on principal component analysis x₁ and on local jets x₂. In hypothesis of independence between these two vectors, the class conditional density function may be written as:
ƒ(x₁, x₂|Cᵢ)=ƒ(x₁|Cᵢ)ƒ(x₂|Cᵢ)

Thus, as described above, the maximum likelihood of the test image feature is examined to make a decision and one can write:

$$P [ {C}_{i} \vert {x}_{1} , {x}_{2} ] is proportional to f ( {x}_{1} \vert {C}_{i} ) f ( {x}_{2} \vert {C}_{i} ) \vert$$

To summarize, with reference to the classifier using MoG, two separate trainings are made using the two set of features and two separate tests. Then, the class conditional densities are multiplied the class with the highest value is chosen.

Without Hypothesis

This approach was suggested by the good performance achieved modeling data with MoG. No assumptions are made on features.

The training set are divided into two sets. The first set is used to train separately the classifier using the two different kinds of features and to get f(xᵢ|cⱼ,θₙ), where x is a feature vector and θₙ is the model used. The model is referred to as the technique for feature extraction, namely the method based on PCA or local jets. Then, for each image of the second training set, the vector is computed:
(P[C₁|data, θ_{pca}], . . . , P[C₁|data, θₗⱼ], . . . )|
and one may model with a MoG these probability vectors of all the images belonging to each class.

In test, given an image, the two feature vectors and the vector of probabilities may be computed. Then, a decision is made by computing the maximum likelihood of this vector in the last training model.

In this way, the performance of each classifier is taken into account and more weight is implicitly given to the “expert's” response with better performance in the given class (even if in test the true class to which the particle belongs is not known).

The drawback of this method, is that it requires a lot of training data because it needs to build MoG models. Because the data set may be quite small, the parameter estimation cannot be very precise and thus, the performance is not improved (see the experimental section).

Boosting

The multi-class extension of AdaBoost called “AdaBoost.M1”, [26] [15] was studied.

Given a weak learner, let be X the set of input data and Y the set of the possible labels for the samples in X. The learner receives examples (xᵢ,yᵢ), with

$${x}_{i} ∈ X and . {y}_{i} ∈ Y \vert$$

examples are chosen randomly according to some fixed but unknown distribution P on X×Y. The goal is to learn to predict the label y given an instance x. It is assumed that the sequence of N training examples is (x₁,y₁), . . . , (x_{N},y_{N}). At each iteration or round, the weak learner generates hypothesis which assigns to each instance one of the K possible labels. At the end, the boosting algorithm presents an output hypothesis

$${h}_{f} \text{:} X \to Y \vert$$

with a lower error rate. It is required that each weak hypothesis has a prediction error of less than ½. The error correcting output code technique [25] does not have this constraint but it is slightly more difficult. The classifier described herein has an error rate below 15% and so, this constraint is not a problem.

For any predicate

$$p , ⊢ p ⊣ \vert ,$$

to be 1 if p holds and 0 otherwise.

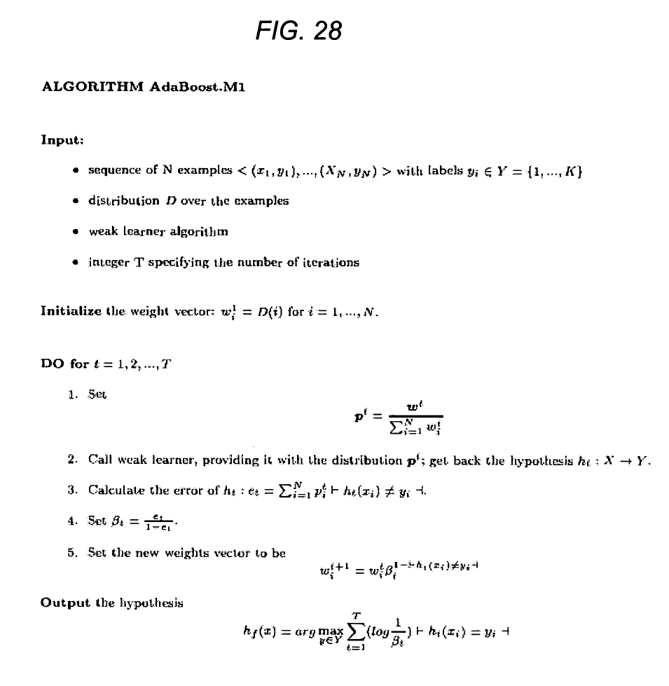

The general algorithm may be summarized in pseudo-code in FIG. 28.

One may attempt to boost the performance of the Mixture of Gaussians classifier described above with reference to the previous algorithm.

One must decide how to provide a classifier with the distribution p at each round. One could go inside EM algorithm and modify the weights of the sum in the log-likelihood computation. However, it seems simpler to extract from each class set a number of images proportional to the class weight. Note that in this way, p becomes a distribution over the classes and not any more over the single training images. This method may also have another drawback because the MoG estimation needs a lot of examples to find convergence. Thus, one may have to reduce the number of training images belonging the best classified categories and on the other hand, keep a sufficient number for the MoG estimate. Thus, an implementation of AdaBoost.M1 may provide a slightly different version.

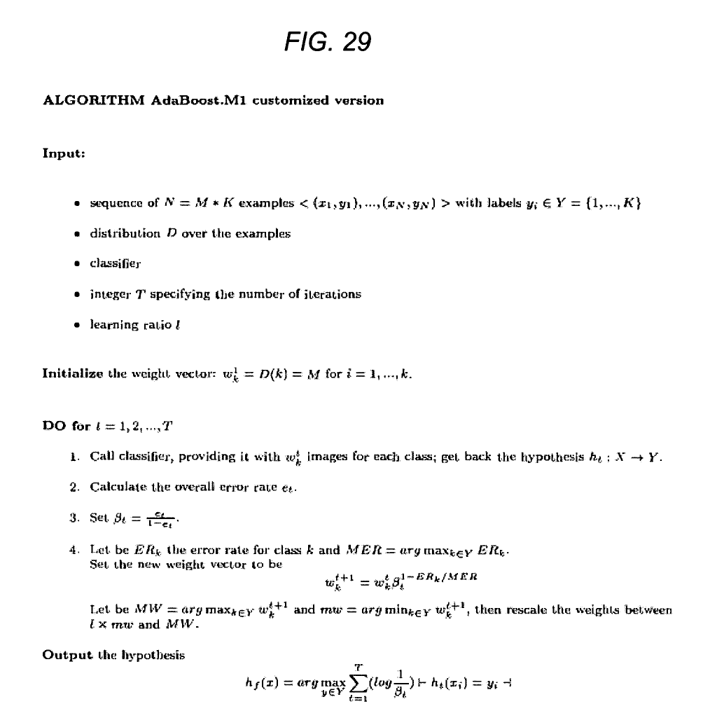

The weights may be chosen in a way that allows the worst classified class to train on all of the maximum number of images while the best classified class has available a minor number of images; however this number is sufficient for the estimate of parameters in the MoG. In particular, at each round, the minimum weight is decreased if the class is still the best classified, while the maximum weight is always equal to the maximum if the class is still the worst classified. The pseudo-code of this customized version of AdaBoost.M1 is shown in FIG. 29.

In the experimental section herein, it may be shown that this method is able to achieve a slightly better performance than the one of the basic classifier.

Support Vector Machines

The support vector machine (SVM) approach does not attempt to model the distribution of features but it works directly on them.

One may start considering the simplest case of a linear machine trained on separable data, [10] [7]. The training input data are a set of labeled features {xᵢ,yᵢ}, i=1, . . . , l; yᵢ ∈ {−1, 1}, x; ∈ [symbol] . Suppose one has some hyper plane which separates the positive from the negative examples. The points x which lie on the hyperplane satisfy, w·x+b=0 normal to the hyperplane. Let be d₊ and d₋ the shortest distance from the separating hyperplane to the closest positive and negative example respectively. For the linearly separable case, the support vector algorithm looks for the separating hyperplane with the largest margin, defined as d₊+d₋. Thus it can be written that each training point satisfies:

$$\begin{matrix} & \begin{matrix}{x}_{i} \cdot w + b \geq + 1 , & for {y}_{i} = + 1\end{matrix} \\ & \begin{matrix}{x}_{i} \cdot w + b \leq + 1 , & for {y}_{i} = - 1\end{matrix} \\ that is , & \\ & {y}_{1} ( {x}_{i} \cdot w + b ) - 1 \geq 0 ∀ i\end{matrix} \vert$$

Choosing a proper scale for w and b, one can have points for which the equality above holds and; d₊, d₋=1/∥w∥|ed support vectors. Their removal would change the solution and they live in the hyperplanes

$${H}_{1} \text{:} {x}_{i} \cdot {{w}_{} + b}_{⋓ ⊥} = + 1 , {H}_{2} \text{:} {x}_{i} \cdot {\tilde{w}}_{} + b = - 1 \vert$$

It can be shown that in order to find w and b, one may have to solve a quadratic optimization problem. Once these parameters are found, given a test point x, one can simply determine on which side of the decision boundary x lies and assign the corresponding class label.

If the training data are not separable, one may have to introduce positive slack variables ξᵢ, i=1, . . . , l in the previous equations which become:

$$\begin{matrix}{x}_{i} \cdot w + b \geq + 1 - {ξ}_{i} , & for {y}_{i} = + 1 \\ {x}_{i} \cdot w + b \leq + 1 + {ξ}_{i} , & for {y}_{i} = - 1 \\ & {ξ}_{i} \geq 0 ∀ i\end{matrix} \vert$$

Thus, for an error to occur, the corresponding must exceed unity, so

$${\sum }_{i} {ξ}_{4} \vert$$

is an upper bound on the number of training ei ξᵢ s. It can be shown [7] that the quadratic programming problem can be solved introducing a new user defined parameter C in order to assign the desired penalty to errors. An interpretation of C and support vectors is that only support vectors exert a force on the decision sheet and C is an upper bound on this force.

Because a support vector machine only realizes a linear classifier, non-separable data are projected in a very high dimensional feature space in which the data become separable, and this means that in the original feature space the classifier becomes highly non-linear.

The only way in which training data appear in the optimization problem is in the form of dot products, xᵢ·xⱼ one can map the data in another (generally higher dimensional space) H using the function Φ: Φ: [symbol] [symbol] H

Now, the training depends only on the data through dot products in H, i.e. on Φ(xᵢ)·Φ(xⱼ)| one be interested to find a kernel function K such that K(xᵢ, xⱼ)=Φ(xᵢ)·Φ(xⱼ) in order to use only a function.

The most powerful and simple kernels are:
linear:

$$K ( {x}_{i} , {x}_{j} ) = {x}_{i} \cdot {x}_{j} \vert$$
polynomial of order p:

$$K ( {x}_{i} , {x}_{j} ) = {( {x}_{i} \cdot {x}_{j} + 1 )}^{p} \vert$$
Gaussian radial basis function (RBF) of parameter
   σ: K(xᵢ,xⱼ)=e−|xᵢ−xⱼ|²/2σ²

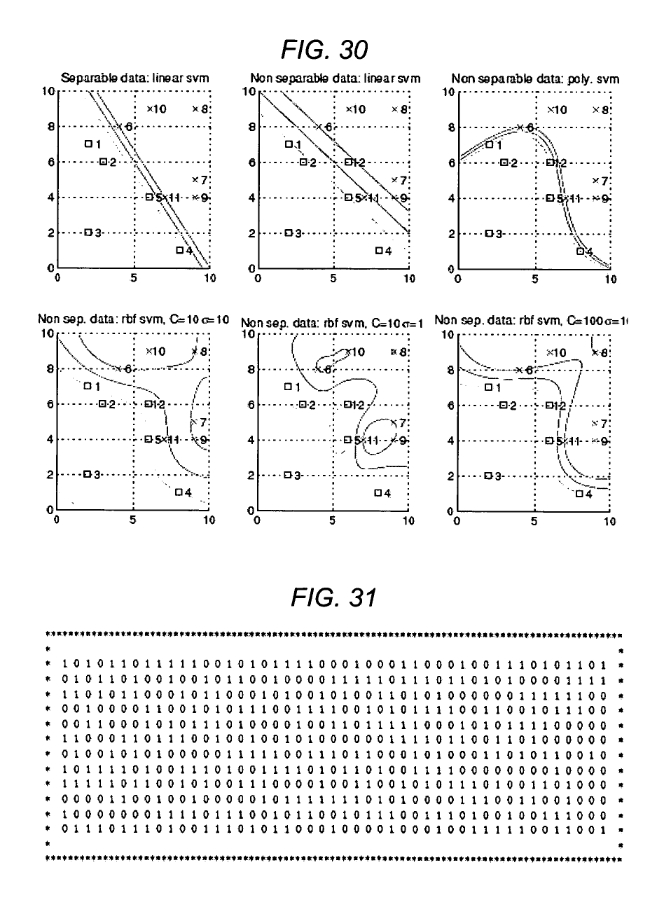

FIG. 30 illustrates a simple example of binary classification using these kernels for different values of their parameters. Thus, FIG. 30 illustrates an example of classification using SVM for separable and non-separable synthetic data with different choices of kernel. Support vectors may be drawn as boxes. The decision boundary is more shattered for high values of degree of polynomial kernel, low values of σ in RBF kernel, high values of C. A balance between generalization and performance in training set has to be found.

Finally, below is a discussion of how to use this algorithm to solve a multiclass classification problem [12] (such a method may also be applied to the AdaBoost algorithm): one may use an error-correcting output codes technique.

Each class is assigned a unique binary string of length n called codeword. Then, n binary functions are learned, one for each bit position in these binary strings. During training for an example of class i, the desired outputs of these n binary functions are specified by the codeword for class i. New values x are classified by evaluating each of the n binary functions to generate an n-bit string s. This string is then compared to each of the k codewords, and x is assigned to the class whose codeword is closest (e.g., using Hamming distance) to the generated string s.

The error-correcting code should satisfy the two properties:
each codeword should be well-separated in Hamming distance from each of the other codewords
each bit-position function should be uncorrelated with the functions to be learned for the other bit positions

For experiments the error-correcting code may be derived by T. Dietterich's collection of code matrices [3]. FIG. 31 illustrates error correction codes used in SVM experiments to solve the multiclass classification problem: 12 classes, codewords with 47 bits.

The SVM algorithm may not be implemented but a software may be downloaded [4] in Matlab written by Anton Schwaighofer (2002). Its main features are:
Except for the QP solver, all parts are written in plain Matlab.
Extension to multi-class problems via error correcting output codes is included.
   Experiments

This section is divided into two parts. First, a description of the method to tune the parameters is provided followed by the final results.

Tuning

This section describes which parameters must be optimized and the optimization method. The urine database is referred to because the reasoning is the same for the pollen database.

MoG

The classifier using MoG, no matter the kind of feature used, needs to be optimized with respect to the choice of:
1. dimension of the feature space after application of Fisher's Linear Discriminants;
2. number of components in each mixture;
3. structure of the covariance matrix of each component; and
4. initial condition for EM algorithm used to estimate the mixture.

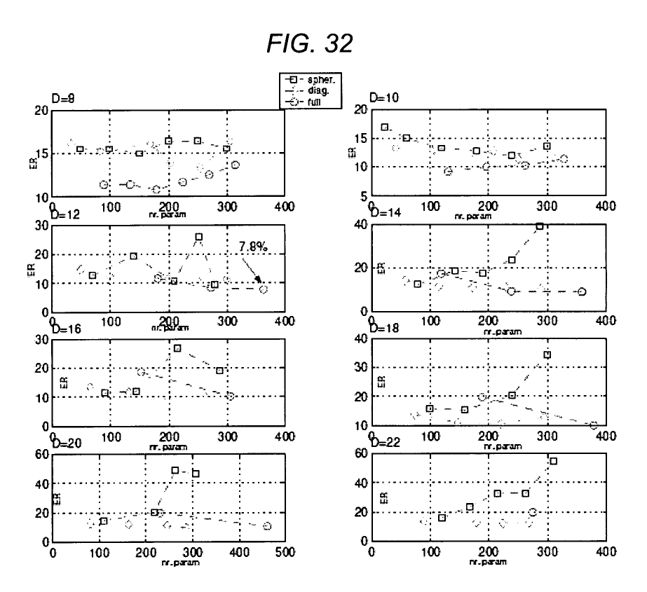

In order to achieve the best reliable (and hopefully also the best) result, one may proceed with a systematic method. The urine database has available twelve classes and nearly one thousand images for each category. Mucus class has only 546 images, Nhyal 860 and NSE 999. One wants to work during this optimization with the same number of images in test and training for each class. Accordingly, the total number of available images in all classes is divided in two sets: the first 500 will be used for only training purposes while the complementary set for only test. From the former set, 30 images may be randomly extracted to build a validation set. All training models will be shown in the following pages, are done on the rest 470 images of training set and the evaluation of the performance is done on the validation set. Then, when optimal parameters are found, a final test is done on 30 randomly chosen images of the test set. As can be seen from the curves of error rate (see FIG. 32 and following figures), the performance is strongly dependent not only on the number of parameters used in the MoG (see TABLE 5) but also on the kind of parameters. In this regard, FIG. 32 illustrates a classifier using MoG and features based on local jets: ER as a function of the parameter number for different choices of feature space dimensions and structure of covariance matrix.

For example, in previous simulations it can be found that even if one keeps constant the number of parameters, the error rate sometimes increases if one elects to increase the dimension of the space instead of increasing the number of Gaussians in the mixture. For this reason, a lot of simulations may be run for different values of the feature space dimension. While this value is maintained constant, several numbers of components are tried for different structures of covariance matrix (spherical, diagonal and full).

In order to estimate a parameter, at least 3 points are needed. In training, 470 images are used and so a search should stop when a number of 150 parameters is reached. On the other hand, an analysis should be exhaustive and so simulations up to 300 parameters should be performed. However, when simulations are run with spherical covariance matrix, there are usually more then ten Gaussians (the maximum value was 25). Unfortunately, EM sometimes does not find convergence for a so high number of Gaussians even if the number of parameters is acceptable. Indeed, upon checking the starting number of points assigned to each Gaussian by “k-means” algorithm, it was found that some Gaussian has only a few points (less than ten) and it is likely that these functions easily collapse in a single point or can give rise to local minima in the error function which may give poor representations of the true distribution. This assessment is strengthened by some warning (probability of data given the model nearly zero) of EM program and by the results of a test on the validation set.

As can be seen in FIG. 32, there are some irregularities in the trend of spherical covariance matrix because for a high number of Gaussians EM doesn't find a model for one or two classes (giving an acceptable global error rate but for those categories the error rate is 100%!).

When experiments are run for the combination of classifiers these situations are avoided by stopping the program at a lower number of Gaussians in the spherical case because to look for a stable system and a reasonable estimate of P[Cᵢ|data, modᵢ].

The classifier using features based on PCA needs to work with dimensions of space multiple of six because each IPCs is split in three parts and it may be required to get the same number of dimensions coming from image and spectrum principal analysis. For this reason, the following experiments are done for a number of dimensions equal to 12, 18 and 24. The same argument is applied to the combination of classifiers.

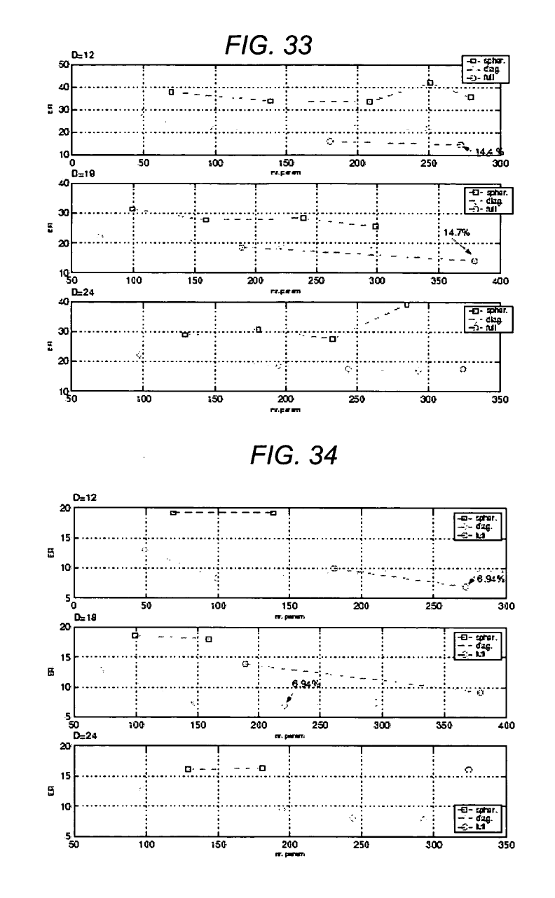

In view of the above, FIG. 33 illustrates a classifier using MoG and feature based on image and spectrum principal components: ER as a function of the parameter number for different choices of feature space dimensions and structure of covariance matrix. Similarly, FIG. 34 illustrates a classifier using MoG and a combination of feature in independence hypothesis: ER as a function of the parameter number for different choices of feature space dimensions and structure of covariance matrix.

The combination of classifiers modeling their outcomes needs a special care. As illustrated in FIG. 34, there are two trainings. in previous studies it was found that a good MoG for the second training (dummy test to model outcomes) has two spherical Gaussians in each MoG. Such a model is used to optimize the first training and then with these good values another simulation is run to find good parameters for the second training. This new analysis confirms the previous results. Thus, the first training may be chosen to run on 470 images per class (the full data training set) and then to select from this set 170 images to perform the second training.

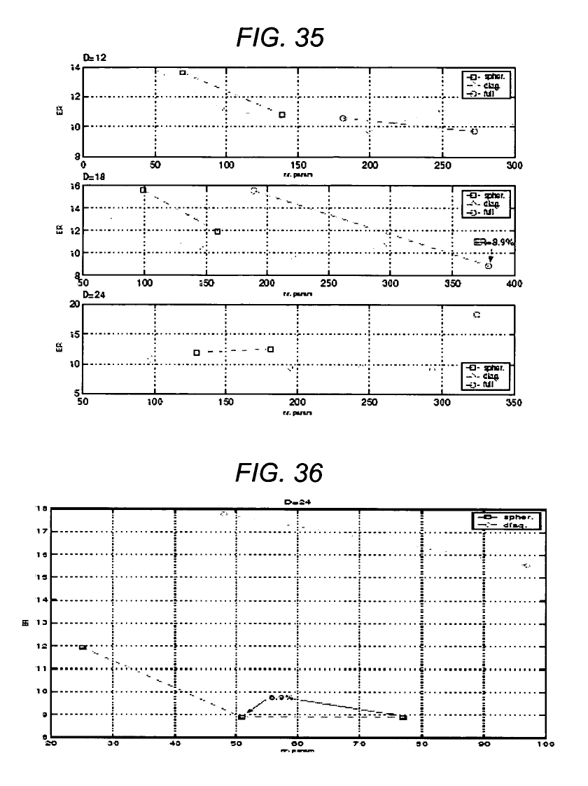

FIG. 35 illustrates a classifier using MoG and making a combination of experts' response, first training: ER as a function of the parameter number for different choices of feature space dimensions and structure of covariance matrix. Further, FIG. 36 illustrates a classifier using MoG and making a combination of experts' response, second training (or modeling of results in dummy test): ER as a function of the parameter number for different choices of feature space dimensions and structure of covariance matrix.

TABLE 6
Best parameters found for MoG in urine database.

| Features | gauss. | dimens. |
|---|---|---|
| Local jets | 4 full | 12 |
| PCA | 3 full | 12 |
| Comb. feat. (indep.) | 6 diag. | 18 |
| Experts' | 2 full | 18 |
| combination | | |

The last set of experiments has the goal to find the optimal initial conditions for EM. A program of the invention calls some functions of NETLAB package to estimate the MoG (e.g., “gmm”, “gmminit” and “gmmem”). The first routine uses the Matlab function “randn” (generator of normally distributed random numbers), the second one calls “randn” and “rand” (generator of uniformly distributed random numbers). The initial state of these two functions is a vector with two components for “randn” and with 35 components for “rand”. So, for each mixture (that is for each class model), three vectors may be found. One hundred experiments may be run for each classifier leaving initial conditions free. At each iteration, these vectors are stored and the performance on the validation set. At the end, the initial state is forced to that value which gave the lowest ER on the validation set.

Obviously, the number of Gaussians, kind of covariance matrix and number of space dimensions are the ones found in the previous simulations.

The results of these experiments are shown in the form of scatter plots. At each iteration in which a different initial condition was chosen, a training model was estimated and the test was done on 30 images per class randomly extracted from the training set, test set and validation set. One can note the generally stronger correlation between the test and validation error rate versus test and training error rate.

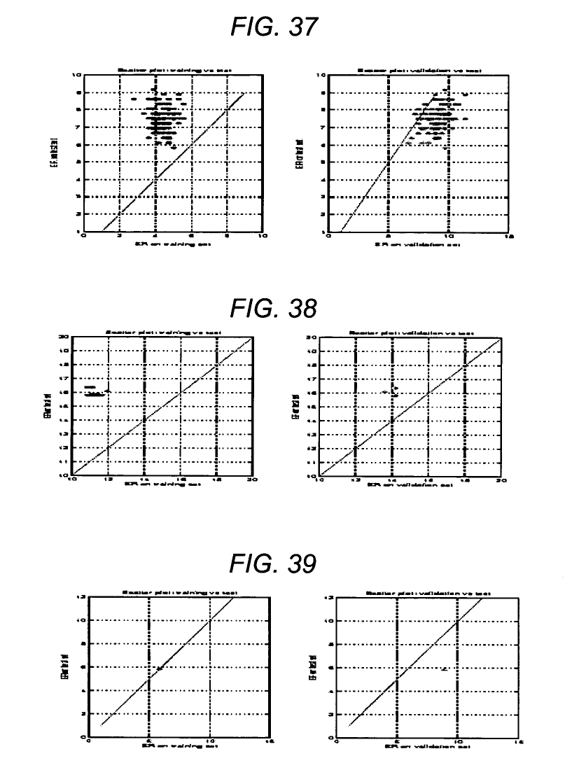

FIGS. 37-39 illustrate such scatter plots in accordance with one or more embodiments of the invention. In FIG. 37, a classifier is displayed using MoG and feature based on local jets: each point in each scatter plot shows the error rate; 100 experiments were run with different initial conditions of random number generator.

FIG. 38 shows a classifier using MoG and feature based on image and spectrum principal components: each point in each scatter plot shows the error rate; 100 experiments were run with different initial conditions of random number generator (a lot of points are overlapped).

FIG. 39 shows a classifier using MoG and making a combination of experts' response: each point in each scatter plot shows the error rate; 100 experiments were run with different initial conditions of random number generator (all points are overlapped).

In view of the above, in the classifier that combines the experts' outcomes, as illustrated in FIG. 39 (a similar situation happens also in FIG. 38), there is no dependence of the performance on the initial conditions of the random number generator. This is not surprising since the maximum number of iterations of EM was chosen equal to one thousand and in this classifier the parameters of only two spherical Gaussians are estimated. For the classifier using a feature based on image and spectrum principal components, three states are possible.

AdaBoost

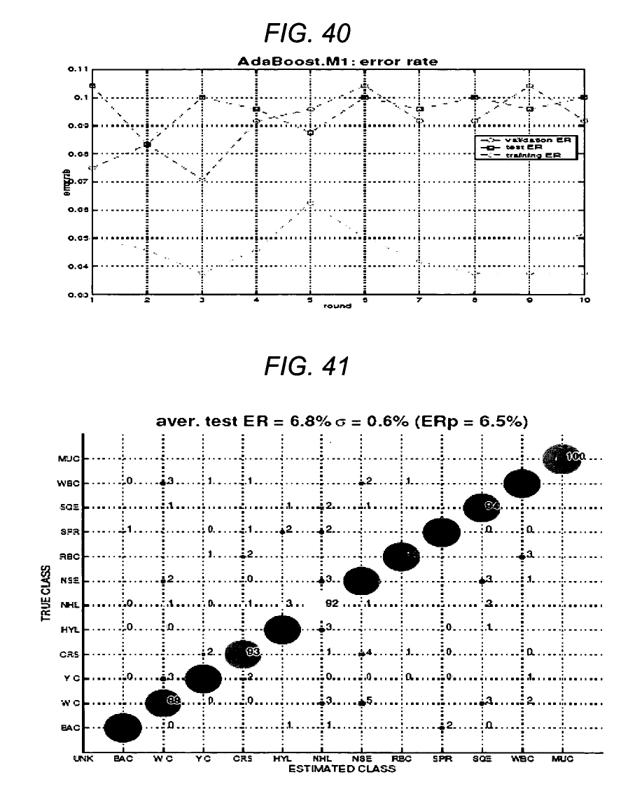

500 images may be considered from each class and 20 of them are extracted randomly for validation. The complementary set is used for the final test. In FIG. 40, the training, validation and test error rates are shown at each round. Thereafter, the 10 hypothesis is combined in the final one as suggested in the pseudocode described above. Thus, FIG. 40 illustrates an AdaBoost.M1: test, validation and training error rate as a function of the number of rounds. Training was done on 480 images, each test on 20 images (randomly chosen).

SVM

The parameters one aims to find are:
1. kernel type;
2. number of dimensions of feature space after reduction with Fisher's Linear Discriminants;
3. parameter C; and
4. parameter of kernel, i.e. degree of polynomial kernel or width of Gaussian radial basis function.

From first experiments, it turns out that RBF kernels is able to achieve a lower error rate than polynomial kernel. So, this specification may only refer to the results of RBF kernel. The results are summarized in TABLES 7-11. The experiments are done taking 500 images for each class and extracting (only once) 30 images for validation purpose.

TABLE 7
SVM with RBF kernel and penalty of errors C = 1: error rate as
a function of the width σ and dimension D of feature space.
The best performance is highlighted in thick black letters.

| σ | D 10 | D 12 | D 14 | D 16 | D 18 | D 20 | D 22 |
|---|---|---|---|---|---|---|---|
| 0.25 | 10.83 | 11.67 | 12.78 | 15.00 | 16.94 | 19.17 | 0.28 |
| 0.5 | 9.17 | 8.89 | 9.17 | 9.44 | 10.56 | 10.00 | 10.83 |
| 1 | 10.83 | 10.83 | 10.00 | 10.56 | 11.67 | 10.56 | 11.11 |
| 1.5 | 11.39 | 11.11 | 11.39 | 12.22 | 12.78 | 11.94 | 12.22 |
| 2 | 11.94 | 12.22 | 12.50 | 13.06 | 13.06 | 13.33 | 13.89 |
| 3 | 14.17 | 13.61 | 13.61 | 14.44 | 14.17 | 15.00 | 14.44 |
| 4 | 15.28 | 14.72 | 15.28 | 15.00 | 15.83 | 15.83 | 14.72 |
| 5 | 16.11 | 15.56 | 15.56 | 16.11 | 16.39 | 16.11 | 17.22 |
| 10 | 18.33 | 16.94 | 18.61 | 18.61 | 18.33 | 18.89 | 18.33 |
| 15 | 20.28 | 18.33 | 18.33 | 18.89 | 19.44 | 19.44 | 20.28 |
| 20 | 22.50 | 19.44 | 20.00 | 20.56 | 20.83 | 21.11 | 21.94 |
| 50 | 26.11 | 23.06 | 24.44 | 25.28 | 27.22 | 28.33 | 28.89 |

TABLE 8
SVM with RBF kernel and penalty of errors C = 5: error rate as
a function of the width σ and dimension D of feature space.
The best performance is highlighted in thick black letters.

| σ | D 10 | D 12 | D 14 | D 16 | D 18 | D 20 | D 22 |
|---|---|---|---|---|---|---|---|
| 0.25 | 12.50 | 12.78 | 12.50 | 15.28 | 16.39 | 18.33 | 19.44 |
| 0.5 | 10.83 | 10.00 | 8.61 | 9.44 | 9.44 | 8.89 | 10.28 |
| 1 | 10.00 | 10.56 | 9.72 | 9.72 | 9.44 | 8.61 | 9.17 |
| 1.5 | 11.39 | 10.83 | 11.39 | 10.56 | 11.67 | 10.00 | 9.72 |
| 2 | 11.39 | 11.67 | 11.39 | 11.39 | 12.22 | 10.83 | 10.83 |
| 3 | 11.67 | 11.39 | 11.94 | 11.94 | 12.22 | 11.67 | 11.94 |
| 4 | 11.94 | 11.94 | 12.22 | 13.33 | 12.78 | 12.22 | 12.22 |
| 5 | 11.94 | 10.28 | 9.44 | 10.28 | 11.39 | 11.11 | 10.56 |
| 10 | 12.78 | 11.39 | 11.67 | 11.94 | 12.78 | 12.78 | 13.06 |
| 15 | 13.33 | 13.06 | 12.78 | 13.89 | 14.17 | 13.89 | 14.72 |
| 20 | 14.44 | 14.44 | 14.72 | 14.72 | 14.72 | 14.44 | 15.28 |
| 50 | 20.56 | 18.06 | 18.89 | 18.89 | 19.44 | 20.00 | 20.56 |

TABLE 9
SVM with RBF kernel and penalty of errors C = 2: error rate as
a function of the width σ and dimension D of feature space.
The best performance is highlighted in thick black letters.

| σ | D 10 | D 12 | D 14 | D 16 | D 18 | D 20 | D 22 |
|---|---|---|---|---|---|---|---|
| 0.25 | 10.56 | 11.39 | 11.94 | 11.67 | 13.61 | 13.33 | 16.11 |
| 0.5 | 13.33 | 10.28 | 10.00 | 10.56 | 12.78 | 11.11 | 11.39 |
| 1 | 10.83 | 10.28 | 11.39 | 9.44 | 8.89 | 9.72 | 9.17 |
| 1.5 | 10.00 | 9.72 | 9.44 | 9.44 | 10.83 | 8.89 | 9.17 |
| 2 | 10.28 | 8.89 | 10.00 | 10.28 | 10.56 | 8.61 | 9.72 |
| 3 | 10.28 | 10.00 | 11.11 | 11.67 | 10.28 | 10.56 | 9.72 |
| 4 | 11.11 | 10.83 | 12.22 | 13.06 | 11.11 | 10.83 | 10.83 |
| 5 | 11.67 | 11.39 | 11.94 | 12.50 | 11.67 | 11.94 | 11.94 |
| 10 | 12.22 | 12.50 | 11.39 | 12.50 | 12.78 | 13.61 | 13.06 |
| 15 | 13.89 | 13.06 | 13.33 | 12.78 | 14.17 | 14.44 | 15.00 |
| 20 | 15.00 | 14.72 | 13.33 | 12.50 | 14.44 | 14.44 | 15.00 |
| 50 | 15.83 | 16.67 | 17.78 | 18.33 | 18.89 | 18.89 | 19.00 |

TABLE 10
SVM with RBF kernel and penalty of errors C = 50: error rate
as a function of the width σ and dimension D of feature space.
The best performance is highlighted in thick black letters.

| σ | D 10 | D 12 | D 14 | D 16 | D 18 | D 20 | D 22 |
|---|---|---|---|---|---|---|---|
| 0.25 | 12.78 | 12.22 | 12.50 | 14.17 | 15.56 | 18.61 | 19.17 |
| 0.5 | 12.22 | 10.28 | 9.44 | 10.83 | 10.28 | 10.83 | 10.28 |
| 1 | 10.28 | 10.00 | 9.72 | 9.72 | 10.56 | 9.17 | 8.61 |
| 1.5 | 10.28 | 10.28 | 10.83 | 9.17 | 10.56 | 9.17 | 9.72 |
| 2 | 10.56 | 10.00 | 9.72 | 10.00 | 9.44 | 9.72 | 9.72 |
| 3 | 9.72 | 10.28 | 11.39 | 9.72 | 10.83 | 9.44 | 10.00 |
| 4 | 10.83 | 10.83 | 11.94 | 10.28 | 10.56 | 10.28 | 10.28 |
| 5 | 11.39 | 10.83 | 11.39 | 11.67 | 11.94 | 10.00 | 11.39 |
| 10 | 12.22 | 12.22 | 12.50 | 11.94 | 12.78 | 12.50 | 12.78 |
| 15 | 13.33 | 12.50 | 12.78 | 13.61 | 12.78 | 13.89 | 13.89 |
| 20 | 14.17 | 13.89 | 14.72 | 15.00 | 13.89 | 15.00 | 14.44 |
| 50 | 16.94 | 16.11 | 16.39 | 16.39 | 17.22 | 18.61 | 18.89 |

TABLE 11
SVM with RBF kernel and penalty of errors C = 100: error rate
as a function of the width σ and dimension D of feature space.
The best performance is highlighted in thick black letters.

| σ | D 10 | D 12 | D 14 | D 16 | D 18 | D 20 | D 22 |
|---|---|---|---|---|---|---|---|
| 0.25 | 11.67 | 11.67 | 14.17 | 15.00 | 16.11 | 18.89 | 18.89 |
| 0.5 | 8.89 | 8.89 | 10.28 | 10.28 | 11.11 | 11.11 | 11.67 |
| 1 | 8.06 | 8.33 | 7.50 | 7.50 | 8.33 | 8.89 | 8.89 |
| 1.5 | 7.50 | 7.78 | 7.50 | 5.83 | 8.06 | 6.67 | 8.61 |
| 2 | 6.94 | 7.50 | 7.78 | 6.94 | 6.67 | 6.39 | 7.78 |
| 3 | 7.50 | 8.61 | 7.50 | 8.06 | 7.22 | 7.50 | 7.50 |
| 4 | 8.61 | 8.06 | 7.78 | 8.06 | 8.06 | 7.22 | 7.50 |

Final Experiments

The final experiments show the final results for urine and pollen database. In order to assess the performance, a confusion matrix is built. Each row is the class to which the test images belong, the columns are the classes to which the classifier assigns the test images. Thus, in the intersection between the j-th row and the i-th column, the following estimate may be found:

$$P [ outcome ∈ {C}_{i} \vert image ∈ , {C}_{j} ] = \frac{nr . of items of {C}_{j } classified as belonging to {C}_{i}}{total nr . of t ested images belonging to {C}_{j}} \vert$$

In order to evaluate the overall performance the error rate defined is computed as:

$$ER = \frac{nr . of not correctly classified test images }{total nr . of test images} \vert$$

Because not all the categories in the databases have the same number of images, an uneven number of images may be trained and tested for class. Then, a new error rate is defined that is the normalized sum of single class error rates

$$ERp = \frac{1}{K} {\sum }_{j = 1}^{K} \frac{nr . of misclassified test images belonging to {C}_{j}}{total nr . of test images belonging to {C}_{j}} \vert$$

For example, when one considers the whole system for pollen recognition, it can be seen that the classifier has to analyze particles of junk. The ratio between pollen and junk particles is 1 out of 10. If 100 pollen patches and 1000 junk patches are tested, it is possible that the classifier has an ER=10% which can be considered quite good. However, it is possible that most of junk particles are classified as junk and most of pollen particles are misclassified, the ER simply can not say if the less numerous classes are well classified. Instead when one considers also the ERp you can check if the error is evenly or proportionally distributed among the categories.

Urine Database

First, the classifier is considered using a mixture of Gaussians with the different kinds of features studied.

During the tuning of parameters, data belonging to the training and validation set was focused on. Below illustrates some results of experiments done in the following way:
1. training and test using the full data set available and with a different number of images per class; attention is placed with putting, in the training set, all of the images belonging to the previous training and validation set, and choosing randomly (from the complementary set) 10% of the total amount of images for test; the result will be the average of 10 experiments;
2. one hundred (100) experiments are run on the fill data set available; at each iteration, about 10% of images are randomly extracted for test purposes.

FIGS. 41-44 illustrate the graphical representation of a confusion matrix using the first method for the four different kinds of classifiers based on MoG modeling.

Note that the numbers in the plot are in percentage with respect to the total number of images tested for a certain class. Moreover, the average result is shown on 10 experiments.

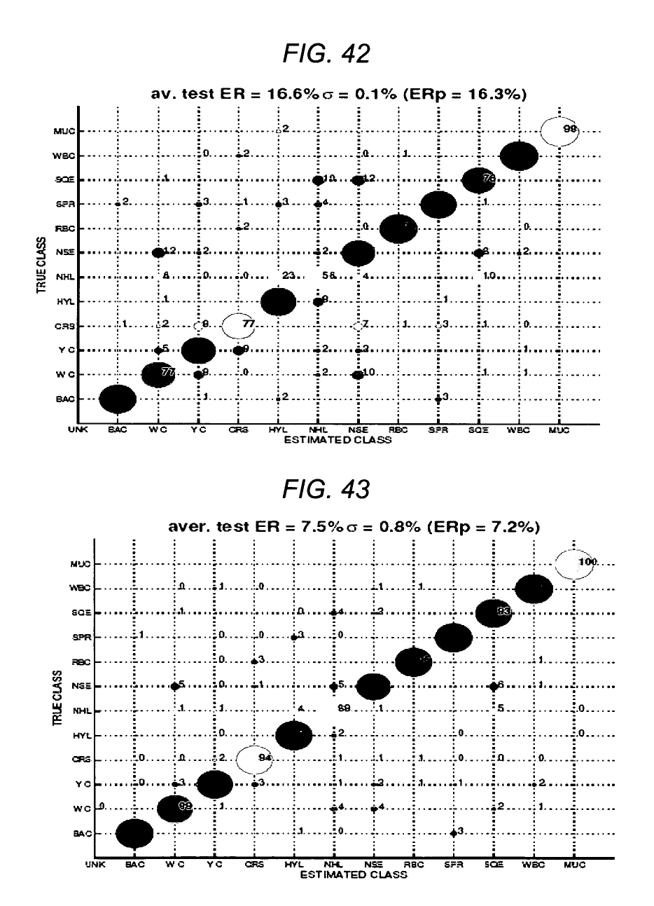

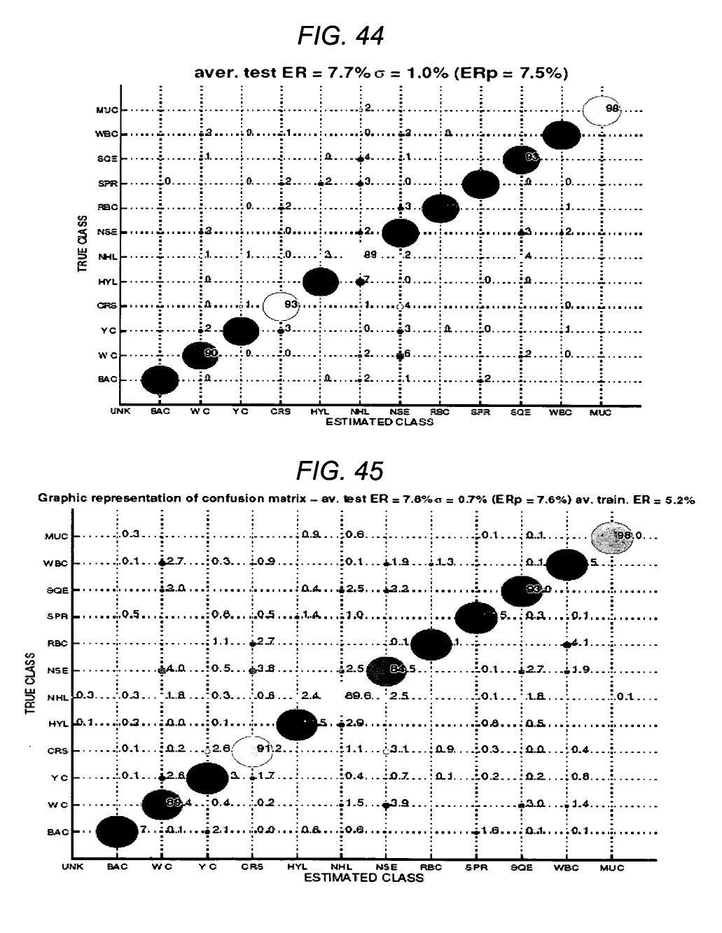

FIG. 41 illustrates a classifier using MoG and feature based on local jets: averaged confusion matrix for experiments in which 90% of images in the full data set is used for training and 10% is randomly extracted for test; there is no overlapping between the test items and previous trained images. FIG. 42 illustrates a classifier using MoG and feature based on image and spectrum principal components: averaged confusion matrix for experiments in which 90% of images in the full data set is used for training and 10% is randomly extracted for test; there is no overlapping between the test items and previous trained images. FIG. 43 illustrates a classifier using MoG and feature combination in indep. hyp.: averaged confusion matrix for experiments in which 90% of images in the full data set is used for training and 10% is randomly extracted for test; there is no overlapping between the test items and previous trained images. FIG. 44 illustrates a classifier using MoG and making a combination of experts' response: averaged confusion matrix for experiments in which 90% of images in the full data set is used for training and 100% is randomly extracted for test; there is no overlapping between the test items and previous trained images.

The experiments with the classifier that combines the experts' outcomes, need a comment. The subdivision of the training set is chosen for the first and second training. When 7/9 of the training set is used for the first training and 4/9 for the second one (so, there is a partial overlap of 2/9) an ER equal to 9.8% results; a second time all the images of the training set are taken for the first training and half of them are extracted randomly from this set for the second training resulting in an ER equal to 8%. The trend was confirmed by the last experiment, shown in FIG. 44, which used (for both trainings) all the images available in the training set; to produce an ER of 7.7%. Even if the second training models the training outcomes (or the training errors), the general performance is improved.

The best result is achieved with the classifier using features based on local jets, its error rate is equal to 6.8% (the proportional error rate is 6.5% ).

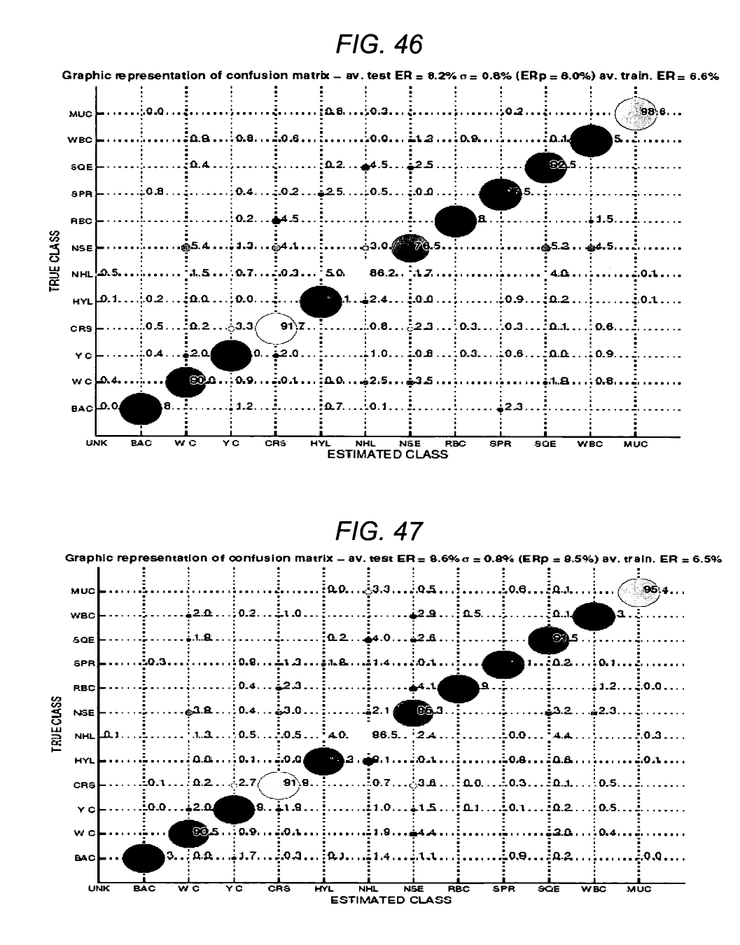

FIGS. 45-47 shows the average results of 100 experiments on the full data set taking randomly 10% of images for test. In this regard, FIG. 45 illustrates a classifier using MoG and feature based on local jets: averaged confusion matrix of test errors for 100 experiments in which 90% of images in the full data set is used for training and 10% is randomly extracted for test. FIG. 46 illustrates a classifier using MoG and feature combination in indep. hyp.: averaged confusion matrix of test errors for 100 experiments in which 90% of images in the full data set is used for training and 10% is randomly extracted for test. FIG. 47 illustrates a classifier using MoG and making a combination of experts' response: averaged confusion matrix of test errors for 100 experiments in which 90% of images in the full data set is used for training and 10% is randomly extracted for test.

Why were 100 experiments performed? Because it is desirable to obtain a reliable estimate of the average ER with a low standard deviation. A brief overview of the theoretic study for the choice of the experiment number is described herein. If x is a stochastic binary variable with alphabet Aₓ={0, 1} and P[x=1]=p(1)=p, it is easy to find that the mean of x is mₓ=p and its variance σₓ²=p(1−p). This variable can be interpreted as the outcome of the following event: “the classifier takes the right decision on the test image”.

If one considers n stochastic binary variables and all these variables have the same parameter p and are independent, the sum variable may be defined as

$$y = {\sum }_{i = 1}^{n} {x}_{i} \vert$$

which is a binomial one with parameters n and p; its mean and variance are:

$${m}_{y} = np = {nm}_{x ;} {σ}_{y}^{2} = np ( 1 - p ) = n {σ}_{x}^{2} \vert$$

Let be z=y/n, then
m_{z}=m_{y}/n=p=mₓ, and σ_{z}²=σ_{y}²/n²=σₓ²/n

Y describes the statistic of n decisions of the classifier on n test images, and z the performance when considering n test images. It can be noted that increasing n you can decrease σ_{z}².

To have a reliable result for the performance of the classifier the results may be averaged on many experiments. Given the (estimated) performance, if the number of images to test and the number of experiments is properly chosen, the variability of the error rate can be controlled.

Let be m the number of experiments to run and

$$w = \frac{1}{m} {\sum }_{i = 1}^{m} {z}_{i} \vert$$

then it can be shown that

$${m}_{w} = {m}_{z} = {m}_{x} and {σ}_{w}^{2} = \frac{1}{m} {σ}_{z}^{2} = \frac{1}{m} {σ}_{x}^{2}$$

Finally, one can approximately estimate the standard deviation of the ER in the experiment doing the following hypothesis: if the probability to misclassify an image is nearly p=0.1 (even if it depends on the belonging class), m is equal to 100 and n is about 100*12 (in practice it is less), then

$${σ}_{w} = \frac{{σ}_{x}}{\sqrt{mn}} ≃ \frac{\sqrt{9 \cdot {10}^{- 2}}}{\sqrt{12 \cdot {10}^{4}}} ≃ 7 \cdot {10}^{- 3}$$

With this choice of parameters, one can be confident to find an ER with a standard deviation of about 0.7% (below 1%).

This result is confirmed by experiments as illustrated in FIGS. 45-47. For example, as can be seen in FIG. 45, the confusion matrix has a high symmetry with respect to the diagonal. For example, 2.5% of Hyal are classified as Nhyal and vice versa, 4% of NSE are classified as white cell clumps and 3.9% of these particles are classified as belonging to NSE class.

The best performance is again achieved using the classifier with features based on local jets, the error rate is equal to 7.6%. If one looks at FIG. 45, the best classified class is mucus with an ER equal to 2%; the worst classified particle is NSE. Most errors are done between white cell clumps and NSE, white cell clumps and squame epithelius, hyal and nhyal.

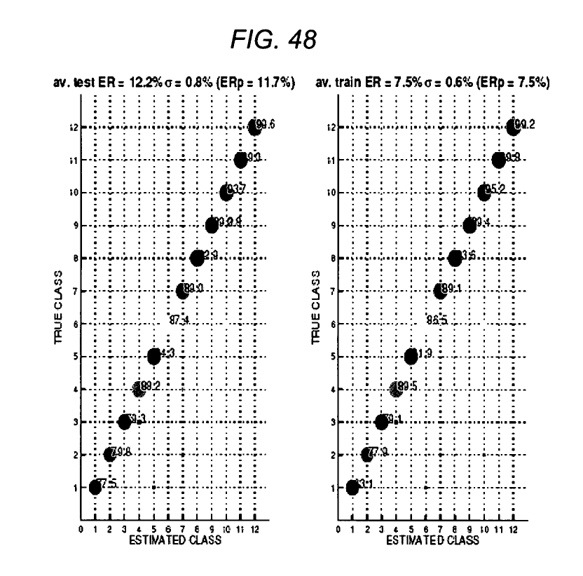

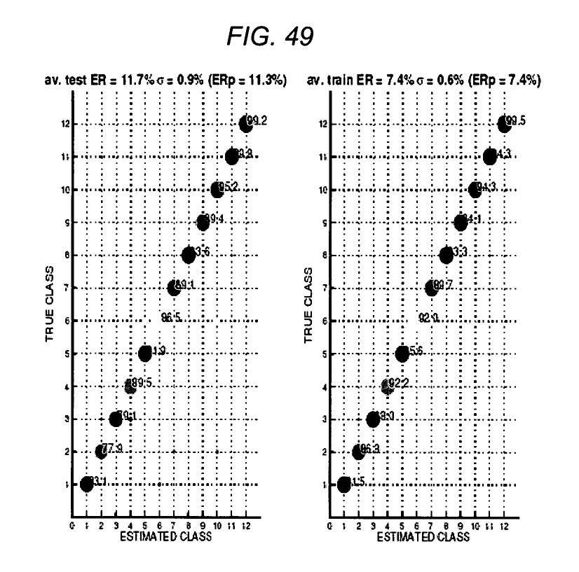

The results of AdaBoost inspired algorithm may also be shown. With the hypothesis found during the experiments of FIG. 40, a test is run on 10% of the images in the data set (not belonging to previous training or validation set). In FIG. 48 and FIG. 49, the averaged performance on 10 experiments is shown for the standard classifier (that is taking only the first hypothesis) and for the boosted one. As illustrated, there is an improvement of 0.5% in the error rate.

FIG. 48 illustrates test and training confusion matrices for the original classifier using MoG and local jet based features. The training is performed on 480 images and the test on 10% of full data set. The result is averaged on 10 experiments. In this figure and in the following ones, the classes are identified with numbers in accordance to TABLE 1. The numbers in the diagonals are the percentages of correct classification in each class.

FIG. 49 illustrates AdaBoost.M1: test and training confusion matrices. The training is performed on 480 images and the test on 10% of full data set. The result is averaged on 10 experiments.

Taking the best achieved performance on validation set, experiments are run using SVM following the methodology above-mentioned for AdaBoost. Unfortunately, an error rate of 12.2% was achieved in test and below 2% in training. This is caused by training data overfitting. Parameters must be chosen for SVM to find a sort of balance between accuracy attained on a particular training set, and the capacity of the machine to learn the general properties of the data. With this choice of parameters, the system seems to have a little generalization. For this reason, other “good” but sub-optimal values may be chosen for the parameters and the best performance is reported in FIG. 50. There is still a big gap between the performance on training and test set however there is an improvement of 0.5% and 1% in the absolute and proportional error rate with respect to the classifier using MoG. Thus, FIG. 50 illustrates SVM with rbf kernel C=5, σ=1 in a 20 dimensional feature space: test and training confusion matrices. The training is performed on 500 images and the test on 10% of full data set. The result is averaged on 10 experiments

Pollen Database

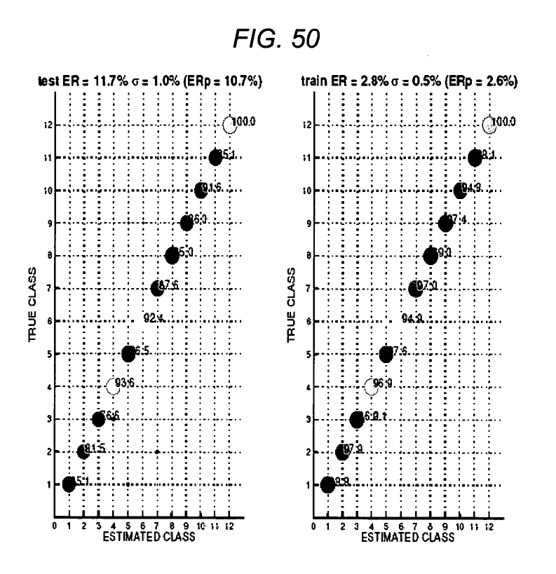

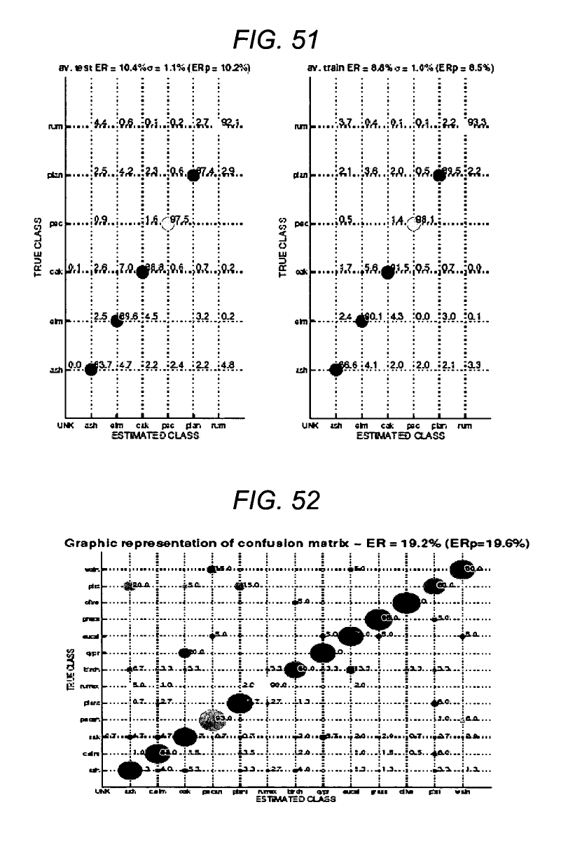

The below description shows the performance of a classifier using MoG with feature based on local jets. The database considered is the “pure” pollen database and the six most numerous categories. These classes have more than 1000 images. It turns out from the tuning stage that the best modeling is achieved taking 2 full covariance matrix in each mixture and considering a 20 dimensional feature space. The tuning was done on 500 training images per class from which 30 images were extracted to build a validation set. Then, 100 experiments are run taking from each class the 10% of available images for test and the rest for training, see TABLE 2. Attention is paid to avoid that images belonging to previous training and validation sets are selected in the current test set. The averaged test error rate is 10.2%, see FIG. 51.

FIG. 51 illustrates a test and training confusion matrix for the six most numerous categories of “pure” pollen. The result is averaged on 100 experiments (no overlapping between test set and current or previous training sets). Analogously, an experiment may also be conducted taking the 13 most numerous classes in the “pure” pollen database (see TABLE 2), that is all the categories with more than 100 images and a proportional averaged test error rate around 19% was obtained, see FIG. 52. FIG. 52 illustrates a test confusion matrix for the 13 most numerous categories of “pure” pollen. The result is averaged on 100 experiments (no overlapping between test set and current or previous training sets).

Is it useful to test the classifier on “pure” pollen database? How should these results be read?

In order to answer to these questions, a description of an analysis of pollen database that gives a deep insight on how the “pure” pollen database is related to the air sampled pollen database is provided.

Consider the classes of air sampled pollen database with more than 60 items. These categories are: ash, chinese elm and oak. In the “pure” pollen database these species have more than 1000 images, see TABLE 12 and TABLE 3. A test on a number of images that is the 10% of air sampled pollen database is performed. The experiments done with the classifier using Mog and local jet based features are:
1. train and test on pollen grains captured by air sampler machine;
2. train and test on “pure” pollen grains with a number of images equal to case 1;
3. train on “pure” pollen grains and test on air sampler pollen grains using the same number of images of case 1;
4. train and test on “pure” pollen grains using for training all the remaining part of images in the “pure” pollen database; and

5. train on the same “pure” pollen images of case 4 and test on the air sampled pollen grains of case 3.

TABLE 12
Number of samples used in experiments for pollen
database assessment.

| | Training on | training on | |
|---|---|---|---|
| Class | few samples | many samples | test |
| Ash | 68 | 1275 | 7 |
| Chin. elm | 112 | 1904 | 12 |
| Oak | 60 | 1473 | 6 |

For experiments with few training data, each mixture has two diagonal Gaussians while for experiments with more than 1000 images per class, the distribution of the data is modeled with a mixture of seven diagonal Gaussians.

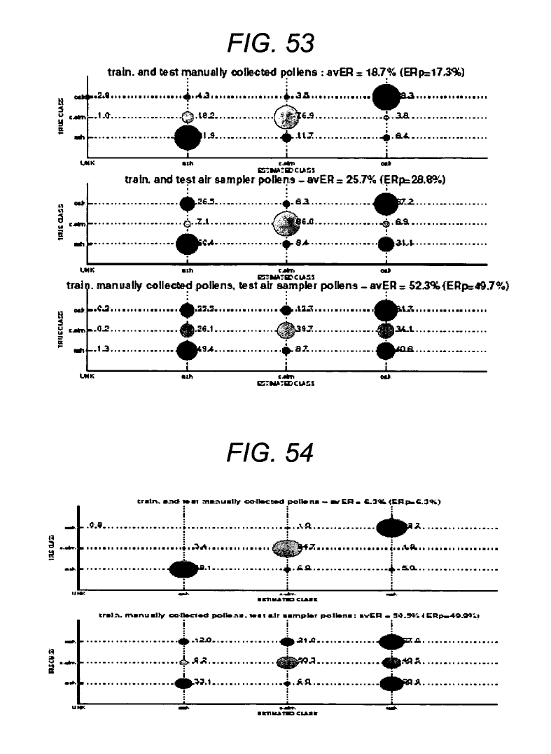

The results are shown in FIGS. 53 and 54. FIG. 53 illustrates a pollen database: confusion matrix when training is done on few samples; the result is an average of 100 experiments. FIG. 54 illustrates a pollen database: confusion matrix when training is done on many images (more than 1000); the result is an average on 100 experiments.

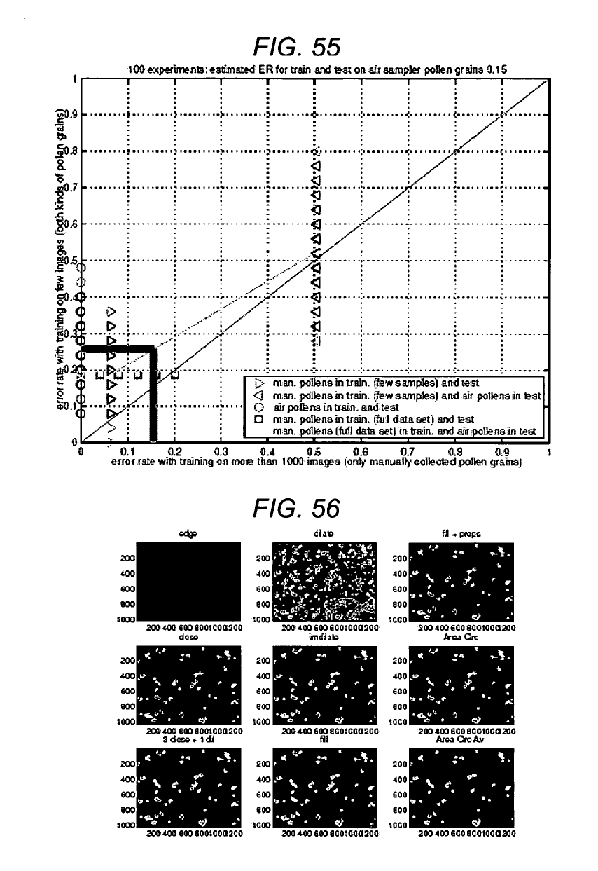

The aim of these experiments is also to find an estimate of the error rate for the same classifier when the training and test are done on air sampled pollen grains and the training has available 1000 images for each class. Since the database of air sampled pollen particles does not have such a quantity of data (see TABLE 2), this error rate may be linearly interpolated from the error rates found in the previous experiments; the promising result of 15% is found as shown in FIG. 55. In this regard, FIG. 55 illustrates a pollen database graphical estimate of the error rate when the training is done using many (more than 1000) images of pollen grains captured by a volumetric spore trap.

Thanks to this analysis, there is an inconsistency between a “pure” pollen and an air sampled pollen database. It is not worthwhile to train with the “pure” pollen images and then to test the air sampled pollen images because the former ones are without junk in the background, less variable in shape, texture and color.

However, training and testing done on a “pure” pollen database will allow one to find a lower bound for the error rate of the classifier using only pollen captured by a spore trap.

The system must be evaluated using air sampled pollen particles in order to have a realistic estimate of the performance of a measurement instrument. However, “pure” pollen images can give an idea of which kind of particles are most difficult to classify.

Moreover, one should observe how much the performance improves with the number of training images; as illustrated in FIGS. 53 and 54, the error rate decreases from 18% to 6% if more images for training are taken when using “pure” pollen images. This fact is also highlighted by the great gap between training and test error rate when few images are taken for training.

Analysis of Errors

In this section, errors made by detector and classifier are analyzed. A goal is to find where the mistakes are made and to understand why they are made. Each section concludes with some ideas to overcome these problems.

Detector

In this section, the detection of pollen in images of tape taken from a volumetric spore trap is discussed.

A detector based on DoG, is not able to detect all particles of interest because there is a quite big difference of dimension among particles belonging to different categories. For example, pine pollen maybe bounded by a square box of side greater than 200 pixels while eucalyptus by a square box of 40 pixel-wide side. So, the sample frequency and the variance of the Gaussian kernel will only be good for grains of a certain size. Moreover, some pollen particles have a very low contrast with respect to the background, because they have no texture. Thus, they can give low values of extrema in the DoG image even if their size is matched by the sample frequency and variance of the Gaussian kernel. Finally, there are a lot of particles in the background that are similar to pollen grains if you only consider information about size and shape as this detector does.

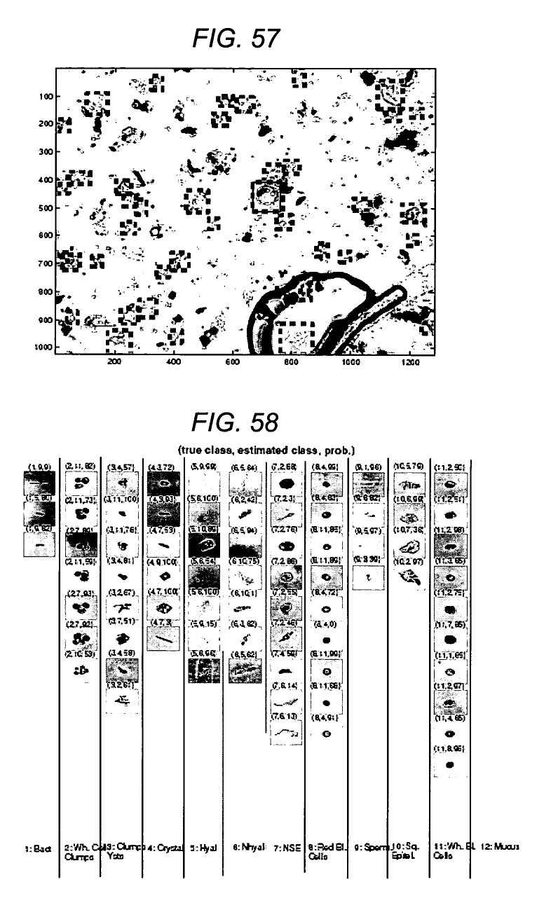

Focusing now on a morphological detector, an example of missed detection is shown in FIGS. 56 and 57. FIG. 56 illustrates a mask computation performed by a morphological detector. FIG. 57 illustrates errors of a morphological detector: the majority of boxes are the automatically detected patches, the black box was selected by the expert.

In this situation, the error was caused by the test on the mean gray level value of pixels fallen inside the region containing the pollen. Because the pollen is quite dark and is attached to a black junk, this region is skipped.

Generally, the kinds of situation that make the detector fail are:
pollen has very low contrast and edge detector is not able to find its contour;
pollen is close to dark junk and the found region contains both of them; and
pollen has dimension or contrast out of range with respect to the fixed thresholds.

The main cause of missed detection is the second one. False alarms are due to the high number of particles in the images with similar statistics to pollen particles. False alarms are typically round objects with the same pollen size but with different texture. Their number could be indirectly reduced increasing the spatial resolution of images, that is, making the wheel of the air sampler machine to rotate faster.

In conclusion, the detector based on DoG seems to be intrinsically limited in its performance because of the high variability in dimension and contrast of pollen grains. The morphological detector could be further improved considering texture inside the regions of interest.

Classifier

In this section, experiments are done on the urine database. The classification errors are analyzed with reference to the Bayesian classifier using feature based on local jets. In particular, experiments are done on full data set taking 10% of images for test. These images are randomly extracted by a set never used for training and validation.

FIG. 58 illustrates the misclassified images with the estimated class and the assigned probability (in percentage). This probability is the probability of the estimated class given the extracted feature vector. It is noted that for most of these images the probability is below 90%. This is a good point because in a real application these items may be assigned to an unknown class for further analysis.

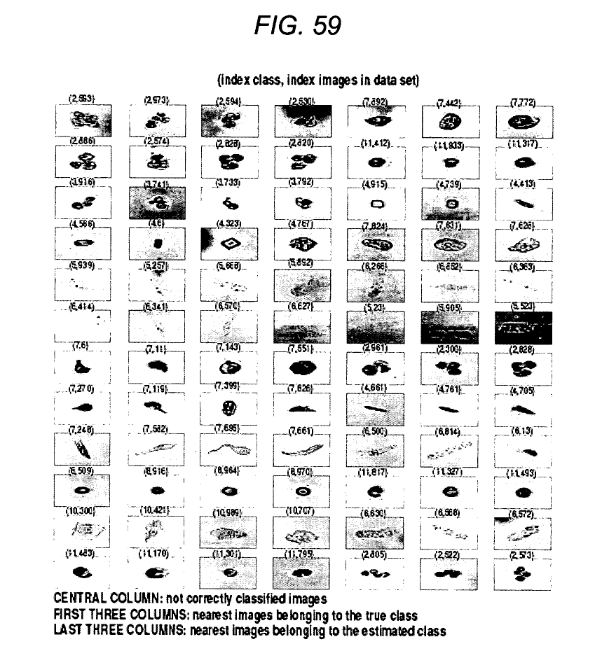

It may be useful to give a deeper insight on the previous misclassification. FIG. 59 shows a collage in which in the central column there are some (misclassified) images of FIG. 58 with assigned high probability. In FIG. 59, the nearest images are in the Euclidean norm of the feature space. In the first three columns there are images belonging to the true class that are the nearest to the one in central column in the Euclidean norm of the feature space (after the projection on FLD). In the last three columns, there are images belonging to the estimated class that are the nearest in the same feature space.

If one follows a row the smooth transition in the features space from one class to the other may be seen; with this interpretation, the misclassified image is on the boundary between the two classes and it can be thought like a transition item.

It may be noted that some errors could be avoided by considering features with information on shape. For example, in the second row of FIG. 59, it seems clear that the average of local jets doesn't allow one to separate clearly the image on the fourth column from the image in the fifth column. But this difficulty may be overcome considering shape and texture of these two images. On the other hand, there are images that are difficult to distinguish because of a certain intrinsic ambiguity of the database.

The difference between the images on the fourth and fifth columns in the intersection with the fifth, sixth, tenth and eleventh row may be questionable. Experts who provided this database may not provide any differences. This intrinsic ambiguity may constitute a lower bound for the error rate of any classifier on this data set and this bound could be found with a test on the human (expert's) error rate on this database.

Analogous analysis can be done on pollen database. Errors are made because features do not capture all the essence of texture in particles and because of the lack of use of color information.

Whole System

This section has for its main aim to combine detector and classifier in order to assess the performance of the whole system. Particles may only be identified in the database of pollen captured by a spore trap and so, this database may be referred to throughout the following discussion.

The morphological detector is considered because it gave good results and it is able to do a good segmentation of the object of interest. It is assumed that for each found pollen, there are 10 false alarms. The first step is to build a database with those patches individuated by this detector. Basically, the detector is run on all images with pollen grains captured by an air sampler machine and each detected patch is put in the proper folder according to the experts' reference list. A new class may be added, called “unknown2”, to gather the false alarm patches. Note that during classification a class “unknown1” may also be used. This is the class where the images may be placed that do not pass the test of the equations discussed above.

In TABLE 13, one can find the number of patches in each category and FIG. 60 shows some patches of this database.

TABLE 13
Pollen database built by morphological detector. Number
of patches in each class.

| GENUS | NUMBER OF ITEMS |
|---|---|
| ash | 72 |
| Chin. elm | 119 |
| oak | 67 |
| Pecan | 5 |
| Cypress | 32 |
| Eucalyp. | 3 |
| Grass | 1 |
| olive | 10 |
| Walnut | 17 |
| pine | 32 |
| liq. amber | 19 |
| alder | 13 |
| Asteraceae | 3 |
| c. myrtle | 3 |
| Chenopod | 19 |
| Ginkgo | 3 |
| Mulberry | 36 |
| palm | 21 |
| Plane t. | 7 |
| Poplar | 5 |
| Sycamore | 17 |
| Umbellif. | 4 |
| False alarms | 8922 |

The Bayesian classifier is applied using features based on local jets. This classifier is one of the best found but may need a lot of images to estimate parameters in the MoG. For this reason, only categories with more than 50 images may be considered, namely: ash, elm, oak and obviously “unknown2”. From the “unknown2” set, a number of images is extracted that is coherent with detector performance. The total number of pollen patches is 258, thus 2580 patches of unknown particles are considered. From each class set, the 10% of images are randomly taken for test, the rest is for training. To summarize, 7 images of ash, 6 of oak, 12 of chinese elm and 250 of “unknown2” class are tested class, see TABLE 14.

TABLE 14
Number of samples used in training and test to assess
the performance of the whole system.

| CLASS | TRAIN. | TEST |
|---|---|---|
| ash | 65 | 7 |
| chin, elm | 107 | 12 |
| oak | 61 | 6 |
| unk. 2 | 2330 | 250 |
| total | 2563 | 275 |

The “unknown2” class may be chosen to model or only the pollen categories may be modeled. In experiments, both solutions may be tried.

The test stage in the classifier is modified to take into account the analysis of “unknown2” particles. Precisely, besides the test on resolution, there is a test on the estimated probability of the class given the image. If this value is too low, then the classifier puts the image in class “unknown1”, the same happens if the image resolution is out of the range of every class.

In the next sections, the experiments done to tune the classifier are described followed by the final results.

Tuning

Two systems are considered: the first one models only the three pollen categories, the second one models also the “unknown2” class. For each of these, one must find:
number of components in the mixture and structure of their covariance matrix;
initial conditions for random number generator used to initialize EM algorithm; and
threshold on probability to decide if an image has to be put in “unknown1” class.

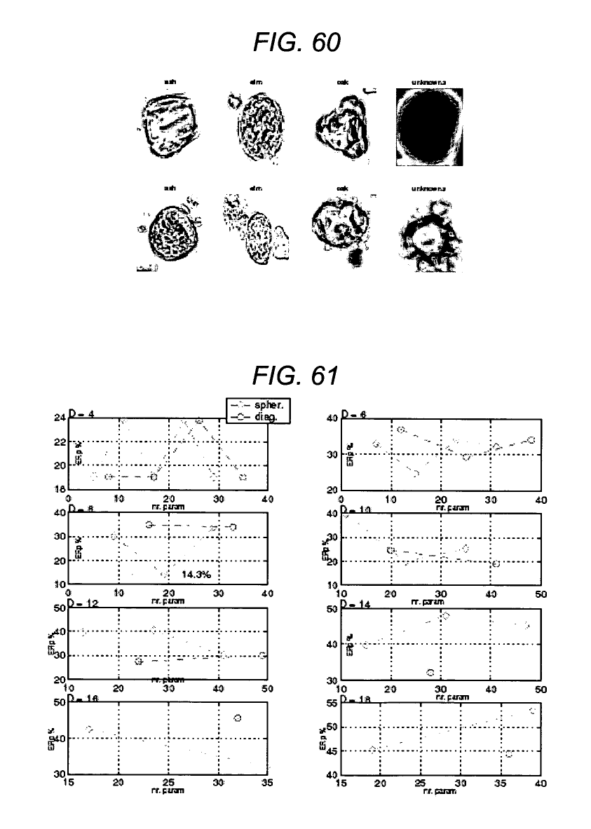

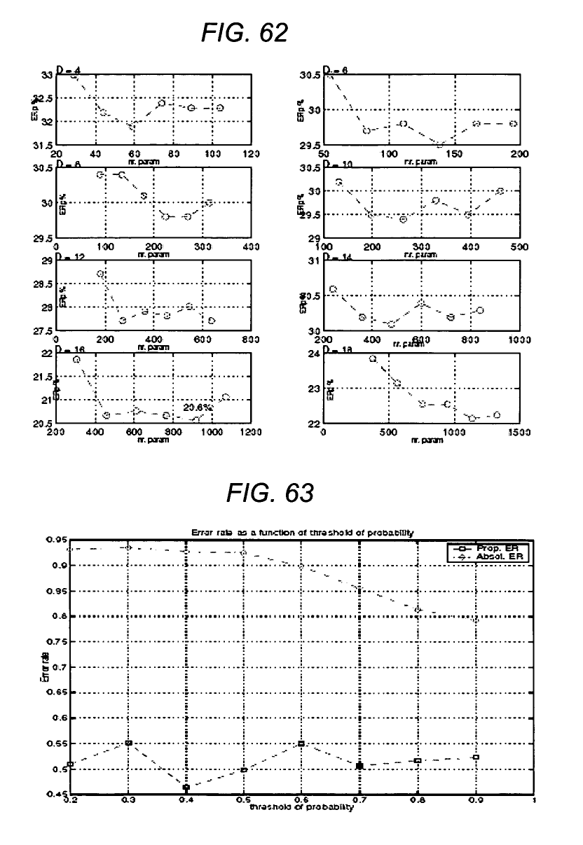

When the optimization of parameters for pollen categories is conducted, only the case of spherical or diagonal structure for the covariance matrix are considered because there may not be enough data to estimate all parameters of a MoG with full covariance matrix components. On the other hand, in the optimization of “unknown2” class, full covariance structure is only computed and a more complex model for this class is computed because of the availability of a lot of images and because this class has presumably a statistics quite broad and complex. In other words, it is likely that points belonging to this class “fill” all of the feature space and do not gather in a single cluster. FIGS. 61 and 62 show the proportional error rate as a function of the number of parameters when the “unknown2” class is not modeled and when it is modeled. In the latter case, only the number of Gaussians in the mixture that model the “unknown2” class may the changed and the pollen categories are described by mixtures with 2 spherical Gaussians. TABLE 15 shows the chosen parameters.

TABLE 15
Parameter chosen for the Bayesian classifier using MoG; the first
row is referred to the classifier that does not model junk class.
The dimension of feature space, the number of components and
their covariance matrix structure is shown for the pollen
models (subscript 1) and for the junk class (subscript 2).

| CLASSIF. | DIMENS. | ng1 | Type1 | ng2 | Type2 | Thres. |
|---|---|---|---|---|---|---|
| only 3 | 10 | 2 | spher. | \ | \ | 0.7 |
| pollen | | | | | | |
| classes | | | | | | |
| 3 pollen | 16 | 2 | spher. | 6 | full | 0.5 |
| classes and | | | | | | |
| unk. | | | | | | |

Note that throughout this section, the proportional error rate as the reference point is different from the previous sections because of the great difference in the number of test images belonging to different categories. Moreover, one considers that an image belonging to “unknown2” class is correctly classified when it is assigned both to class “unknown1” and, if the model exists, to class “unknown2”.

Looking at FIG. 62, and to the absolute error rate here not reported, it may be observed that increasing the dimensions of feature space causes a better classification for the junk class but a worse classification for the pollen categories. Proportional error rate allows one to weight more errors on pollen categories that have less test samples.

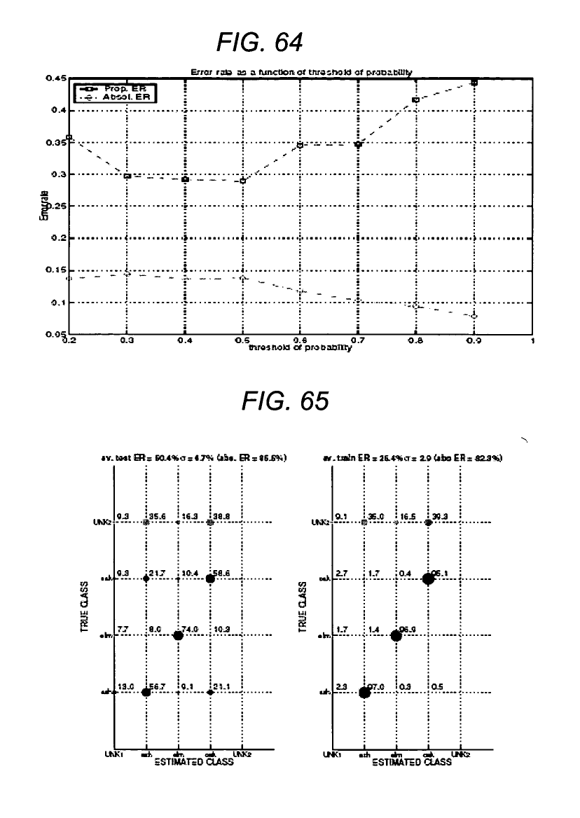

Once also the best initial conditions have been found applying the same method described above, one is ready to estimate the optimal threshold of probability. With the previous optimal parameters found, 10 experiments are run for each choice of threshold; the error rates shown in FIGS. 63-64 are the averaged values. When the system does not model the junk class, the pollen classification is not influenced by the threshold of probability (the line is nearly constant) while the junk class is obviously better identified by a high threshold (the representative line is strongly related to the classification of this class). Indeed as discussed above, it was noted that the classifier is most often wrong when the probability assigned to an image is below 90% and on the other hand, correctly classified images have high probability.

Instead, FIG. 64 shows a behavior more independent from the threshold choice. From this analysis, it may be deduced that it is better to model the “unknown2” class. In FIG. 63, it can be seen that most of the junk particles are misclassified while in FIG. 64, it is noted that this trend is corrected because the absolute error rate, which mainly depends on junk images, is much less than the proportional error rate. This observation will be confirmed by the final results. TABLE 15 summarizes the choice of parameters.

Final Experiments

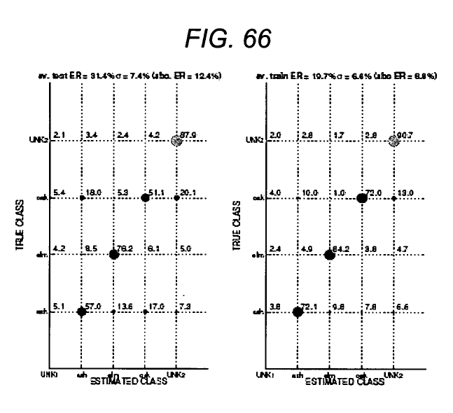

Because of the shortage of images in the database, the database could not be divided in training, validation and test sets. Each time, 10% of the images were randomly selected for test and the rest for training. In order to evaluate the performance of the system, an average of the results of 100 experiments was made. The confusion matrices are shown in FIGS. 65 and 66. FIG. 65 shows a test and training confusion matrix, left and right respectively. The classifier does not model the junk class. Note that almost all junk particles are classified as pollen. FIG. 66 shows a test and training confusion matrix, left and right respectively. The classifier models the “unknown2” class.

The performance of the system that models the junk class is much better. In this system, most of false alarms fall in either “unknown1” or “unknown2” classes. Note that the error rate has about the same value of the error rate found in par. Above when air sampled pollen grains were tested with patches selected by the human expert and without the false alarm class. This is possible because the detector is able to do a segmentation comparable to the expert's one and a good model is used for the false alarm category.

The variance of the error rate is quite high, around 7%. For example in the experiments of FIG. 66, the minimum error rate was about 12% while the maximum 48%. This should be interpreted as a lack of reliability of the result because of the shortage of images used in test and in training.

It should also be noted that there is a gap between training and the test error rate. This means that too few images for training may be used and the training data may be overfitted. On the other hand, using more images for training this gap will likely decrease until nearly zero and the final performance will be around or below 10% as predicted by the reasoning done above.

Conclusion

In this section, the performance of the whole system on images of pollen captured by spore trap was described. The detector is able to detect with a probability of 9/10 all pollen grains but it detects also for each pollen ten other particles. With the spatial resolution of the images, this result is quite good.

The next stage is classification. When the system learns to classify using few images for training and it models also the junk class the error rate is about 30%. This result confirms the performance of the same classifier when the expert's patches were used thus, it may be deduced that the automatic segmentation is really good. Moreover, this time the false alarm class may be dealt with but thanks to a more complex model, the best correct classification rate may be achieved on this category: nearly 90%.

Looking at the training error rate and to the estimate done above, this system will likely be able to achieve an error rate of 10% when it will be fed with more images because of the ability to estimate more complex models and the system will have a better generalization of training data.

The experiments are done in conditions which are very close to a real situation. Particularly, the ratio between pollen and junk particles is kept at one out of ten and the classifier receives objects in the right proportion. In this way, the difficulty to select images for training and to classify pollen particles that ate not modeled because their number is too low is overcome.

To give an idea of the current performance, an example may be useful.

Suppose the system receives 50 images with 100 pollen grains. Then 90 of them will be detected and 1000 false alarms will be generated. The classifier receives 1090 patches and will correctly identify nearly 70% of pollen particles. This means that at the end 63 pollen grains will be correctly recognized by the present invention.

Conclusion

The above text describes a system for visual recognition of biological particles. The interest is justified by the need to automatically do microscopic analysis. This is because manual analysis is slow, expensive, not precise and not standardized. Two databases were worked with: the first one is a collection of particles in urine, the second one is a collection of sticky tape images containing many genera of pollen.

The recognition system can be divided into two stages: detection and classification. Detection aims to find points of interest in an image, in which it exists at least a small probability of a useful particle presence represented by those spots. Classification receives patches from the detector; particularly, it has to skip particles that are not of interest and for the rest to determine their category.

A detector was developed based on morphological operations which gave very good results on a pollen database. It is able to detect with a probability 9/10, the 23 species of pollen in sticky tape pictures taken by light microscope. The percentage of false alarm is about 1000%. Looking at the images in the database, this performance seems very good. Even an expert has to focus his attention on nearly 10 points in each image to establish the presence of a pollen. On the other hand, this approach is not general and reprogramming may be needed to apply this detector to another database. Furthermore, this technique is quite slow compared to other ones based on filtering, the analysis of one image takes up 10 seconds on a current 2.5 GHz Pentium processor computer.

The simplest classifier with the best performance is the Bayesian classifier using a mixture of Gaussians to model the training data. A new kind of features was developed based on local jets [9]; these features prove to capture almost all the essence of input images because of the ability to achieve an error rate of about 7.6% on urine database and 10% on 6 kinds of “pure” pollen. This approach is interesting because it is segmentation free, very general and able to handle very low contrast particles.

On the other hand, the system may be further improved using a more complex technique, that extracts, from points in the foreground, information about texture, shape and color.

Moreover in a real application, the system often has to handle particles that are not of interests, for example in pollen recognition most of the patches contain junk particles. This class should be very well classified if a high number of false alarms are admitted in detection. This is a challenge to consider in the design of a good classifier, and in experiments, this class may be taken into account when the pollen classification task is considered.

One contribution of the invention in classification of urine particles relies on the definition of a new set of powerful features that are able to extract information from patches of about 60×60 pixels without any segmentation. Thanks to the simplicity of the system, one is able to classify particles very quickly; nearly 10 ms are required to analyze a particle on a current 2.5 GHz Pentium processor computer.

In pollen recognition, the present invention provides a feasible system for real-time pollen count in which detection and classification are combined to recognize several kinds of pollen. Images taken with a common light microscope are used and the database is built using the common equipment for pollen collection, namely the volumetric spore trap. Unfortunately, the collection of airborne pollen grains is very slow and the available database is still very small. A larger database would allow a better modeling of data and could improve performance. Thus, the importance has been proven experimentally of using pollen particles captured by a spore trap instead of “pure” pollen grains, that is pollen grains taken directly from flowers. Indeed, the former ones are much more variable in size, shape and texture.

In addition, urinalysis could be improved in the future if a general and segmentation free approach is applied, and if customized operations are reserved only in a second stage for the most difficult samples.

Hardware and Software Environment

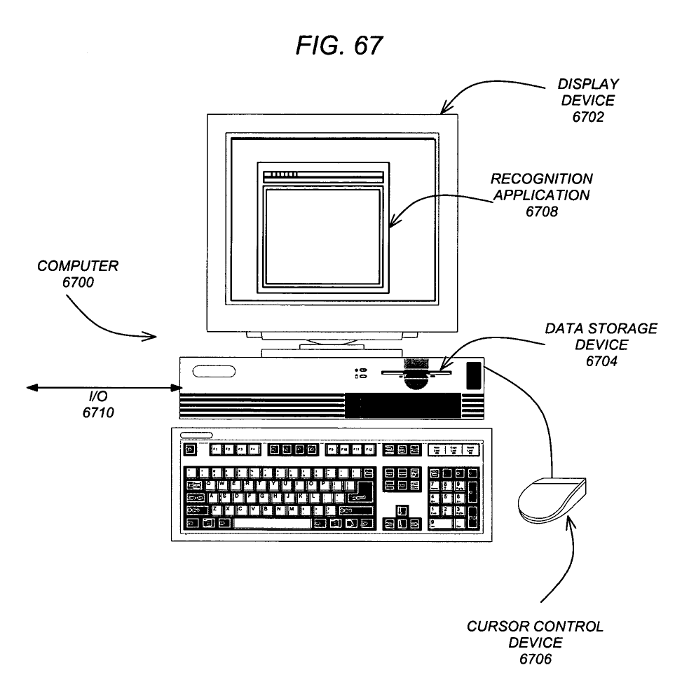

FIG. 67 is an exemplary hardware and software environment used to implement one or more embodiments of the invention. Embodiments of the invention are typically implemented using a computer 6700, which generally includes, inter alia, a display device 6702, data storage devices 6704, cursor control devices 6706, and other devices. Those skilled in the art will recognize that any combination of the above components, or any number of different components, peripherals, and other devices, may be used with the computer 100.

One or more embodiments of the invention are implemented by a computer-implemented recognition application 6708 (e.g., a detector, feature extractor or classifier as described above), wherein the recognition application 6708 may be represented by a window displayed on the display device 6702. Generally, the recognition application 6708 comprises logic and/or data embodied in or readable from a device, media, carrier, or signal, e.g., one or more fixed and/or removable data storage devices 6704 connected directly or indirectly to the computer 6700, one or more remote devices coupled to the computer 6700 via a data communications device, etc. In addition, the recognition application 6708 may process information provided from other aspects of the recognition system of the invention through input/output (I/O) 6710.

Those skilled in the art will recognize that the exemplary environment illustrated in FIG. 67 is not intended to limit the present invention. Indeed, those skilled in the art will recognize that other alternative environments may be used without departing from the scope of the present invention.

Logical Flow

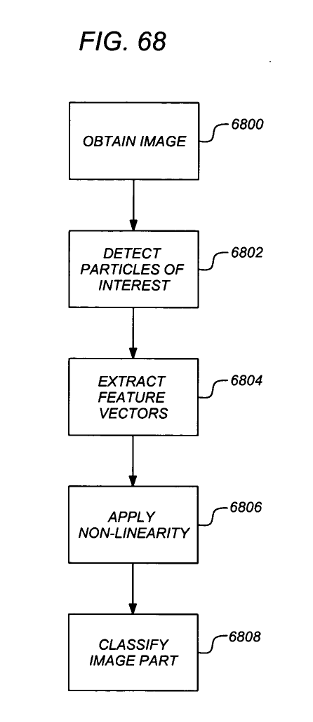

FIG. 68 illustrates the logical flow for implementing a method for automatically recognizing biological particles in accordance with one or more embodiments of the invention. At step 6800, an image comprising biological materials is obtained. Such an image may be of airborne pollen particles obtained using a volumetric spore trap. Alternatively, the image may be images of urine obtained using a light microscope.

At step 6802 one or more parts of the image are detected as containing one or more particles of interest. Such detecting may be based on a filtering approach using a difference of Gaussians (DoG). Further, the detecting may provide a part of the image that is invariant with respect to scale, shift, and rotation.

At step 6804, one or more feature vectors are extracted from each detected part of the image.

At step 6806, one or more non-linearities are applied to each feature vector. Such non-linearities may be applied to an invariant and comprise a piece-wise linear function and a piece-wise quadratic transformation that depends on a range of input invariants. Alternatively, the nonlinearity may divide a range of variation of an invariant signal in each dimension into three parts where one part is sensitive to low values, a second to values around a background mean, and a third sensitive to high values. In a third embodiment, the non-linearity may divide each invariant into three parts—positive, negative, and absolute value followed by adding the background mean to the absolute value. The application of such non-linearities decreases the error rate.

At step 6808, each part of the image is classified into a category of biological particles based on the one or more feature vectors for each part of the image.

### CONCLUSION

This concludes the description of the preferred embodiment of the invention. The following describes some alternative embodiments for accomplishing the present invention. For example, any type of computer, such as a mainframe, minicomputer, or personal computer, or computer configuration, such as a timesharing mainframe, local area network, or standalone personal computer, could be used to implement the method of the present invention.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

### REFERENCES

[1] http://www.aaaai.org

[2] http://medlib.med.utah.edu/WebPath/TUTORIAL/URINE/.

[3] http://web.engr.oregonstate.edu/tgd/software/ecoc-codes.tar.gz.

[4] http://www.cis.utgraz.at/igi/aschwaig/software.html.

[5] The iq200 automated urinalysis system. Brochure IRIS.

[6] C. M. Bishop. Neural Networks for Pattern Recognition. Oxford Univ. Press, 2000.

[7] C. J. C. Burges. A tutorial on support vector machines for pattern recognition. Data mining and Knowledge Discovery, 2(2):121-167, 1998.

[8] M. C. Burl, L. Asker, P. Smith, U. Fayyad, P. Perona, L. Curmpler and J. Aubele. Learning to recognize volcanoes on venus. In Machine Learning, volume 30, pp. 165-194, Boston, February-March 1998. Kluver Academic Publisher.

[9] R. Mohr C. Schmid. Local grayvalue invariants for image retrieval. IEEE Trans. On Pattern Analysis and Machine Intelligence, (19(5)):530-535, 1997.

[10] N. Cristianini and J. Shawe-Taylor. Support vector machines and other kernel-based learning methods. Cambridge University Press, 2000.

[11] J. Dahmen, J. Hektor, R. Perrey and H. Ney. Automatic classification of red blood cells using gaussian mixture densities. In Proc. Bildverarbeitung fur die Medizin, pp. 331-335, Munich, Germany, 2000.

[12] T. G. Dietterich and G. Bakiri. Solving multiclass learning problems via error correction output codes. Journal of artificial intelligence research, 1995.

[13] R. O. Duda and P. E. Hart. Pattern classification and scene analysis. Wiley, New York, 1973.

[14] I. France, A. W. G. Duller, H. F. Lamb and G. A. T. Duller. A comparative study of approaches to automatic pollen identification. BMCV, 1997.

[15] Y. Freund and R. E. Schapire. A decision-theoretic generalization of on-line learning and an application to boosting. In European Conference on Computational Learning Theory, 1996.

[16] K. Fukunaga. Introduction to Statistical Pattern Recognition. Academic Press, second edition, 1990.

[17] J. J. Koenderink and J. van Doorn. Representation of local geometry in the visual system. Biological Cybernetics, (55):367-375, 1987.

[18] M. Langford. Some applications of digital image processing for automation in palynology. Ph.D. Thesis, University of Hull, 1988.

[19] M. Langford, G. E. Taylor and J. R. Flenley. The application of texture analysis for automated pollen identification. Conference on identification and pattern recognition, 1986.

[20] D. G. Lowe. Object recognition from local scale-invariant features. In International conference on computer vision, Corfu, September 1999.

[21] D. G. Lowe. Distinctive image features from scale-invariants keypoints. Submitted for publication, June 2003.

[22] J. R. Flenley M. Langford, G. E. Taylor. Computerised identification of pollen grains by texture. Review of paleibotany and palynology, 1990.

[23] P. Perona. Note on fisher linear discriminants, 2000.

[24] O. Ronneberger, U. Heimann, E. Schultz, V. Dietze, H. Burkhardt and R. Gehrig. Automated pollen recognition using gray scale invariants on 3d volume image data. Vienna, Austria, September 2000. Second European Symposium of Aerobiology.

[25] R. E. Schapire. Using output codes to boost multiclass learning problems. In Machine Learning: Proceedings of the Fourteenth Conference, 1997.

[26] R. E. Schapire. The boosting approach to machine learning an overview. In MSRI workshop on nonlinear estimation and classification, 2002.

[27] E. Grant Smith. Sampling and identifying allergenic pollens and molds. Blewstone Press, San Antonio, Tex., 2000.

[28] A. Torralba and A. Oliva. Statistics of natural image categories. Network: Computation in neural systems, (14):391-412, 2003.

[29] E. L. Vezey and J. J. Skvarla. Computerised feature analysis of exine sculpture patterns. Review of paleibotany and palynology, 1990.

[30] http://www.che.caltech.edu/groups/ref/DataFrameset.html.

[31] C. Harris and M. Stephens. A combined corner and edge detector. Manchester, 1998. 4^{th} Alvey Vision Conference pp. 189-192.

1. A method for recognizing biological particles, comprising:
obtaining an image comprising biological particles;
detecting one or more parts of the image as containing one or more particles of interest;
extracting one or more feature vectors from each detected part of the image;
applying one or more non-linearities to each feature vector; and
classifying each part of the image into a category of biological particle based on the one or more feature vectors for each part of the image.

2. The method of claim 1, wherein the image is obtained using a volumetric spore trap and comprises images of airborne pollen.

3. The method of claim 1, wherein the image is obtained using a light microscope and comprises images of urine.

4. The method of claim 1, wherein the detecting is based on a filtering approach using a difference of Gaussians (DoG).

5. The method of claim 1, wherein the detecting provides a part of the image that is invariant with respect to scale, shift, and rotation.

6. The method of claim 1, wherein one of the non-linearities is applied to an invariant and comprises:
a piece-wise linear function; and
a piece-wise quadratic transformation that depends on a range of input invariants.

7. The method of claim 1, wherein one of the non-linearities is applied to an invariant and comprises dividing a range of variation of a signal of the invariant in each dimension into three parts wherein one part is sensitive to low values, a second part is sensitive to values around a background mean, and a third part is sensitive to high values.

8. The method of claim 1, wherein one of the non-linearities is applied to one or more invariants and comprises:
dividing each invariant into three parts—positive, negative, and absolute value;
adding a background mean to the absolute value.

9. A system for recognizing biological particles comprising:
(a) an image of biological particles;
(b) a detector configured to detect one or more parts of the image as containing one or more particles of interest; and
(c) a classifier configured to:
(i) extract one or more feature vectors from each detected part of the image;
(ii) apply one or more non-linearities to each feature vector; and
(iii) classify each part of the image into a category of biological particle based on the one or mote feature vectors for each part of the image.

10. The system of claim 9, further comprising a volumetric sport trap configured to obtain the image wherein the image comprises images of airborne pollen.

11. The system of claim 9, further comprising a light microscope configured to obtain the image wherein the image comprises images of urine.

12. The system of claim 9, wherein the detector is configured to detect one or more parts of the image based on a filtering approach using a difference of Gaussians (DoG).

13. The system of claim 9, wherein the detector is configured to provide a part of the image that is invariant with respect to scale, shift, and rotation.

14. The system of claim 9, wherein one of the non-linearities is applied to an invariant and comprises:
a piece-wise linear function; and
a piece-wise quadratic transformation that depends on a range of input invariants.

15. The system of claim 9, wherein one of the non-linearities is applied to an invariant and comprises dividing a range of variation of a signal of the invariant in each dimension into three parts wherein one part is sensitive to low values, a second part is sensitive to values around a background mean, and a third part is sensitive to high values.

16. The system of claim 9, wherein one of the non-linearities is applied to one or more invariants and comprises:
dividing each invariant into three parts—positive, negative, and absolute value;
adding a background mean to the absolute value.
